(19) United States
(12) Patent Application Publication
(10) Pub. No.: US 2003/0073846 A1
(43) Pub. Date: Apr. 17, 2003

(54) Aminoalcohol derivatives

(76) Inventors: Kiyoshi Taniguchi, Osaka (JP); Hiroshi Kayakiri, Osaka-shi (JP); Minoru Sakurai, Osaka-shi (JP); Naokaki Fujii, San Francisco (CA); Kenichi Washizuka, Osaka-shi (JP); Hitoshi Hamashima, Osaka-shi (JP); Yasuyo Tomishima, Osaka-shi (JP); Kaori Hamada, Osaka-shi (JP); Nobuhiro Yamamoto, Osaka-shi (JP); Hirofumi Ishikawa, Osaka-shi (JP); Naoko Unami, Osaka-shi (JP); Toshiko Miura, Osaka-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/181,970

(22) PCT Filed: Feb. 26, 2001
(86) PCT No.: PCT/JP01/01442

(30) Foreign Application Priority Data
Feb. 28, 2000 (AU) PQ5850

(51) Int. Cl. C 07D 4 3/02; C 07D 4 1/02; C07D 213/62; C07D 213/78; C07D 209/82; C07D 209/14

(52) U.S. Cl. 546/261; 546/276.7; 546/277.7; 546/300; 548/444; 548/503; 564/344

(57) ABSTRACT

A compound of the formula (I) wherein X [symbol] 1 ? is bond or —O(CH₂)ₘ— (in which m is an integral number of 1, 2 or 3); X₂ is bond, —(CH₂)ₙ—, etc. (in which n is an integral number of 1, 2 or 3); R₁ is hydrogen or amino protective group; R₂ is hydroxy(lower)alkyl or (lower)alkoxy(lower)alkyl; A is phenyl, pyridyl, indolyl or carbazolyl, each of which may be substituted with one or two substituent(s) selected from the group consisting of halogen, hydroxy, lower alkyl, etc.; and B is phenyl or pyridyl, each of which may be substituted with one or two substituent(s) selected from the group consisting of halogen, hydroxy, nitro, etc.; and a pharmaceutically acceptable salt thereof which is useful as a medicament.

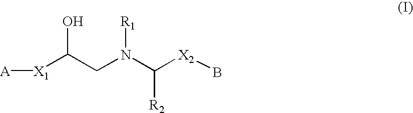

### TECHNICAL FIELD

This invention relates to new aminoalcohol derivatives and salts thereof which are useful as a medicament.

### BACKGROUND ART

Some aminoalcohol derivatives, which have intestinal motility modulating activity, were disclosed in the patent documents, that is WO95/11223 and WO96/32369.

### DISCLOSURE OF INVENTION

This invention relates to new aminoalcohol derivatives and salts thereof.

More particularly, it relates to new aminoalcohol derivatives and salts thereof which act as selective bata-3 (β₃) adrenergic receptor agonists and therefore have gut selective sympathomimetic, anti-ulcerous, anti-pancreatitis, lipolytic, anti-urinary incontinence and anti-pollakiuria activities, to processes for the preparation thereof, to a pharmaceutical composition comprising the same and to a method of using the same therapeutically in the treatment and/or prevention of gastro-intestinal disorders caused by smooth muscle contractions in human beings or animals, and more particularly to a method for the treatment and/or prevention of spasm or hyperanakinesia in case of irritable bowel syndrome, gastritis, gastric ulcer, duodenal ulcer, enteritis, cholecystopathy, cholangitis, urinary calculus and the like; for the treatment and/or prevention of ulcer such as gastric ulcer, duodenal ulcer, peptic ulcer, ulcer caused by non steroidal anti-inflammatory drugs, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis or the like; for the treatment and/or prevention of pancreatitis, obesity, diabetes, glycosuria, hyperlipidemia, hypertension, a therosclerosis, glaucoma, melancholia, depression and the like, and for the treatment and/or prevention of a wasting condition, weight loss, emaciation or the like.

One object of this invention is to provide new and useful aminoalcohol derivatives and salts thereof which have gut selective sympathomimetic, anti-ulcerous, lipolytic, anti-urinary incontinence and anti-pollakiuria activities.

Another object of this invention is to provide processes for the preparation of said aminoalcohol derivatives and salts thereof.

A further object of this invention is to provide a pharmaceutical composition comprising, as an active ingredient, said aminoalcohol derivatives and salts thereof.

Still further object of this invention is to provide a therapeutical method for the treatment and/or prevention of aforesaid diseases in human beings or animals, using said aminoalcohol derivatives and salts thereof.

The object aminoalcohol derivatives of this invention are new and can be represented by the following general formula [I]:

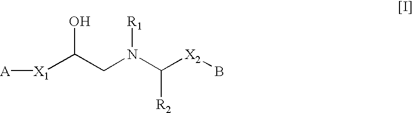

wherein

X₁ is bond or —O(CH₂)ₘ— (in which m is an integral number of 1, 2 or 3);

X₂ is bond, —(CH₂)ₙ— or —CH₂O— (in which n is an integral number of 1, 2 or 3);

R₁ is hydrogen or an amino protective group;

R₂ is hydroxy(lower)alkyl or (lower)alkoxy(lower)alkyl;

A is phenyl, pyridyl, indolyl or carbazolyl, each of which may be substituted with one or two substituent(s) selected from the group consisting of halogen, hydroxy, nitro, lower alkoxy, phenyl(lower)alkoxy, optionally substituted lower alkyl and optionally substituted amino; and

B is phenyl or pyridyl, each of which may be substituted with one or two substituent(s) selected from the group consisting of halogen, hydroxy, nitro, lower alkanoyl, carboxy, (halo(lower)alkyl)sulfonyloxy, optionally substituted amino, optionally substituted lower alkyl, optionally substituted ureido, optionally substituted carbamoyl, (lower)alkoxycarbonyl and lower alkoxy optionally substituted with lower alkoxy, carboxy or phenyl,

(i) provided that when X₁ is —O(CH₂)ₘ— (in which m is an integral number of 1);

X₂ is —(CH₂)ₙ— (in which n is an integral number of 1);

R₁ is hydrogen;

R₂ is hydroxymethyl; and

A is phenyl or pyridyl, each of which may be substituted with one or two substituent(s) selected from the group consisting of halogen, lower alkoxy and lower alkyl; or indolyl,

then B is not phenyl which may be substituted with one or two substituent(s) selected from the group consisting of halogen and lower alkoxy optionally substituted with lower alkoxy or carboxy;

(ii) provided that when X₁ is bond;

X₂ is —(CH₂)ₙ— (in which n is an integral number of 1);

R₁ is hydrogen;

R₂ is hydroxymethyl; and

A is phenyl or pyridyl, each of which may be substituted with one or two substituent(s) selected from the group consisting of halogen and lower alkyl,

then B is not phenyl which may be substituted with one or two substituent(s) selected from the group consisting of halogen, hydroxy, carboxy, nitro and lower alkoxy optionally substituted with lower alkoxy or carboxy;

or a salt thereof.

The object compound [I] or a salt thereof can be prepared by the following processes.

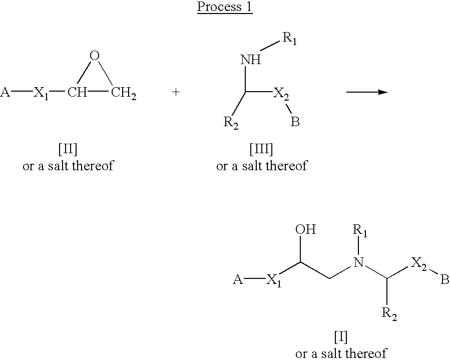

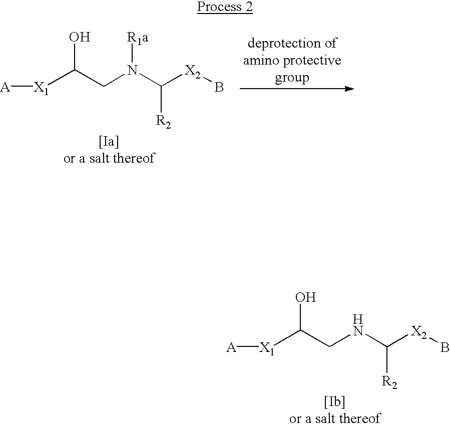

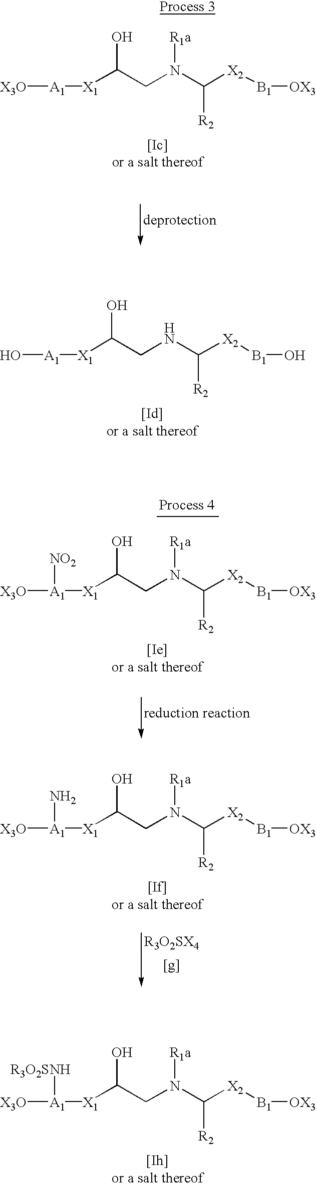

wherein

X₁, X₂, R₁, R₂, A and B are each as defined above,

A₁ is phenyl, pyridyl, indolyl or carbazolyl,

B₁ is phenyl or pyridyl,

X₃ is a hydroxy protctive group,

X₄ is halogen,

R₁a is an amino protective group, and

R₃ is lower alkyl or phenyl optionally substituted with halogen.

In the above and subsequent description of the present specification, suitable examples of the various definition to be included within the scope of the invention are explained in detail in the following.

Substituents for the optionally substituted lower alkyl may include hydroxy, (lower)alkoxycarbonyl, etc.

Substituents for the optionally substituted amino may include phenylsulfonyl, (lower)alkoxycarbonyl, (lower)alkylsulfonyl, formyl, etc.

Substituents for the optionally substituted ureido may include (lower)alkylsulfonyl, etc.

Substituents for the optionally substituted carbamoyl may include lower alkyl, lower alkoxy, (lower)alkylsulfonyl, phenyl, phenylsulfonyl, etc.

The term “lower” is intended to mean a group having 1 to 6 carbon atom(s), unless otherwise provided.

Suitable “lower alkyl” and “lower alkyl” moiety in the terms of “(lower)alkylsulfonylamino”, “hydroxy(lower)alkyl”, “halo(lower)alkyl”, “(lower)alkylsulfonylureido”, “(lower)alkylsulfonyl”, etc. may include straight or branched one having 1 to 6 carbon atom(s), such as methyl, ethyl, propyl, isopropyl, butyl, isobutyl, sec-butyl, tert-butyl, pentyl, 1-methylpentyl, tert-pentyl, neo-pentyl, hexyl, isohexyl and the like.

Suitable “lower alkoxy” and “lower alkoxy” moiety in the terms of “(lower)alkoxycarbonyl”, “(lower)alkoxycarbonyl(lower)alkyl”, etc. may be a straight or branched one such as methoxy, ethoxy, propoxy, isopropoxy, 1-ethylpropoxy, butoxy, sec-butoxy, tert-butoxy, pentyloxy, neopentyloxy, tert-pentyloxy, hexyloxy, and the like.

Suitable “halogen” may be fluoro, chloro, bromo and iodo.

Suitable “halo(lower)alkyl” may include mono(or di or tri)halo(lower)alkyl (e.g., chloromethyl, dichloromethyl, trichloromethyl, bromomethyl, dibromomethyl, tribromomethyl, fluoromethyl, difluoromethyl, trifluoromethyl, 1 or 2-chloroethyl, 1 or 2-bromoethyl, 1 or 2-fluoroethyl, 1,1-difluoroethyl, 2,2-difluoroethyl, etc.), etc.

Suitable “lower alkanoyl” and “(lower)alkanoyl” moiety in the term of “(lower)alkanoylamino” may include formyl, acetyl, propionyl, isobutyryl, valeryl, isovaleryl, pivaloyl, hexanoyl, 3,3-dimethylbutyryl, etc.

Amino protective groups in the context of the invention are the customary amino protective groups used in peptide chemistry. These include benzyloxycarbonyl, 2,4-dimethoxybenzyloxycarbonyl, 4-methoxybenzyloxycarbonyl, methoxycarbonyl, ethoxycarbonyl, tert-butoxycarbonyl, allyloxycarbonyl, phthaloyl, 2,2,2-trichloroethoxycarbonyl, fluorenyl-9-methoxycarbonyl, formyl, acetyl, 2-chloroacetyl, 2,2,2-trifluoroacetyl, 2,2,2-trichloroacetyl, benzoyl, 4-chlorobenzoyl, 4-bromobenzoyl, 4-nitrobenzoyl, phthalimido, isovaleroyl or benzyloxymethylene, 4-nitrobenzyl, 2,4-dinitrobenzyl, 4-nitrophenyl, 4-methoxyphenyl, triphenylmethyl, etc.

Suitable “hydroxy protective group” in the context of the invention may include phenyl(lower)alkyl which may have one or more suitable substituent(s) (e.g., benzyl, 4-methoxybenzyl, trityl, etc.), trisubstituted silyl [e.g., tri(lower)alkylsilyl (e.g., trimethylsilyl, tert-butyldimethylsilyl, etc.), etc.], tetrahydropyranyl, tert-butyl, p-nitrobenzoyl, p-toluene sulfonyl, acetyl and the like.

Suitable salts of the object aminoalcohol derivatives [I] are pharmaceutically acceptable salts and include conventional non-toxic salts such as an inorganic acid addition salt [e.g. hydrochloride, hydrobromide, sulfate, phosphate, etc.], an organic acid addition salt [e.g. formate, acetate, trifluoroacetate, oxalate, maleate, fumarate, tartrate, methanesulfonate, benzenesulfonate, toluenesulfonate, etc.], an alkali metal salt [e.g. sodium salt, potassium salt, etc.] or the like.

Preferred embodiments of the object compound [I] are as follows:

X₁ is bond or —O(CH₂)ₘ— (in which m is an integral number of 1);

X₂ is —(CH₂)ₙ— (in which n is an integral number of 1 or 2);

R₁ is hydrogen;

R₂ is hydroxy(lower)alkyl;

A is phenyl, pyridyl, indolyl, or carbazolyl, each of which may be substituted with one or two substituent(s) selected from the group consisting of halogen, hydroxy, nitro, lower alkoxy, phenyl(lower)alkoxy, lower alkyl, hydroxy (lower) alkyl, (lower)alkoxycarbonyl(lower)alkyl, amino, phenylsulfonylamino optionally substituted with halogen, (lower)alkylsulfonylamino and (lower)alkanoylamino; and

B is phenyl or pyridyl, each of which may be substituted with one or two substituent(s) selected from the group consisting of halogen; hydroxy; nitro; lower alkanoyl; carboxy; (trifluoromethyl)sulfonyloxy; lower alkyl; halo(lower)alkyl; hydroxy(lower)alkyl; carboxy(lower)alkyl; (lower)alkoxycarbonyl(lower)alkyl; amino; (lower)alkoxycarbonylamino; (lower)alkylsulfonylamino; phenylsulfonylamino optionally substituted with halogen; N-(lower alkyl)-N-(phenylsulfonyl)amino optionally substituted with halogen; (lower)alkylsulfonylureido; (lower)alkoxycarbonyl; carbamoyl which may be substituted with one or two substituent(s) selected from the group consisting of lower alkyl, lower alkoxy, (lower)alkylsulfonyl, phenyl and phenylsulfonyl; and lower alkoxy optionally substituted with lower alkoxy, carboxy or phenyl,

(i) provided that when X₁ is —O(CH₂)ₘ— (in which m is an integral number of 1);

X₂ is —(CH₂)ₙ— (in which n is an integral number of 1);

R₁ is hydrogen;

R₂ is hydroxymethyl; and

A is phenyl or pyridyl, each of which may be substituted with one or two substituent(s) selected from the group consisting of halogen, lower alkoxy and lower alkyl; or indolyl,

then B is not phenyl which may be substituted with one or two substituent(s) selected from the group consisting of halogen and lower alkoxy optionally substituted with lower alkoxy,

(ii) provided that when X₁ is bond (in which m is an integral number of 1);

X₂ is —(CH₂)ₙ— (in which n is an integral number of 1);

R₁ is hydrogen;

R₂ is hydroxymethyl; and

A is phenyl or pyridyl, each of which may be substituted with one or two halogen(s),

then B is not phenyl which may be substituted with one or two substituent(s) selected from the group consisting of halogen, hydroxy, carboxy, nitro and lower alkoxy optionally substituted with lower alkoxy.

Further, preferred embodiments of the object compound [I] are as follows:

X₁ is bond or —O(CH₂)ₘ— (in which m is an integral number of 1);

X₂ is —(CH₂)ₙ— (in which n is an integral number of 1 or 2);

R₁ is hydrogen;

R₂ is hydroxy(lower)alkyl;

A is phenyl, pyridyl or carbazolyl, each of which may be substituted with one or two substituent(s) selected from the group consisting of hydroxy, hydroxy(lower)alkyl, nitro, amino, phenylsulfonylamino optionally substituted with halogen, (lower)alkylsulfonylamino and formylamino; and

B is phenyl or pyridyl, each of which may be substituted with one or two substituent(s) selected from the group consisting of halogen, hydroxy, lower alkanoyl, carboxy, (trifluoromethyl)sulfonyloxy, hydroxy(lower)alkyl, carboxy(lower)alkyl, (lower)alkoxycarbonyl(lower)alkyl, amino, (lower)alkoxycarbonylamino, (lower)alkylsulfonylamino, phenylsulfonylamino optionally substituted with halogen, N-(lower alkyl)-N-(phenylsulfonyl)amino, di(lower alkyl)carbamoyl and lower alkoxy optionally substituted with lower alkoxy or phenyl.

Further, preferred embodiments of the object compound [I] are as follows:

X₁ is —OCH₂—;

X₂ is —CH₂—;

R₁ is hydrogen;

R₂ is hydroxymethyl;

A is phenyl which may be substituted with one or two substituent(s) selected from the group consisting of hydroxy, hydroxy(lower)alkyl, nitro, amino, phenylsulfonylamino optionally substituted with halogen, (lower)alkylsulfonylamino and formylamino; and

B is phenyl which may be substituted with one or two substituent(s) selected from the group consisting of halogen, hydroxy, lower alkanoyl, carboxy, (trifluoromethyl)sulfonyloxy, hydroxy(lower)alkyl, carboxy(lower)alkyl, (lower)alkoxycarbonyl(lower)alkyl, amino, (lower)alkoxycarbonylamino, (lower)alkylsulfonylamino, phenylsulfonylamino optionally substituted with halogen, N-(lower alkyl)-N-(phenylsulfonyl)amino, di(lower alkyl)carbamoyl and lower alkoxy optionally substituted with lower alkoxy or phenyl.

More preferred embodiments of the object compound [I] are as follows:

X₁ is —OCH₂—;

X₂ is —CH₂—;

R₁ is hydrogen;

R₂ is hydroxymethyl;

A is phenyl substituted with hydroxy and phenyl-sulfonylamino optionally substituted with halogen; and

B is phenyl substituted with hydroxy or lower alkoxy.

Further, more preferred embodiments of the object compound [I] are as follows:

X₁ is bond;

X₂ is —(CH₂)ₙ— (in which n is an integral number of 1 or 2);

R₁ is hydrogen;

R₂ is hydroxymethyl;

A is phenyl which may have one or two substituent(s) selected from the group consisting of hydroxy, hydroxy(lower)alkyl, amino, (lower)alkylsulfonylamino, phenylsulfonylamino which may be substituted with halogen and formylamino; and

B is phenyl which may have one or two substituent(s) selected from the group consisting of hydroxy, halogen and lower alkoxy.

The processes for preparing the object compound [I] are explained in detail in the following.

Process 1

The object compound [I] or a slat thereof can be prepared by reacting a compound [II] with a compound [III] or a salt thereof.

Suitable salt of the compound [III] may be the same as those exemplified for the compound [I].

The reaction is preferable carried out in the presence of a base such as an alkali metal carbonate [e.g. sodium carbonate, potassium carbonate, etc.], an alkaline earth metal carbonate [e.g. magnesium carbonate, calcium carbonate, etc.], an alkali metal bicarbonate [e.g. sodium bicarbonate, potassium bicarbonate, etc.], tri(lower)alkylamine [e.g. trimethylamine, triethylamine, etc.], picoline or the like.

The reaction is usually carried out in a conventional solvent, such as an alcohol [e.g. methanol, ethanol, propanol, isopropanol, etc.], diethyl ether, tetrahydrofuran, dioxane, or any other organic solvent which does not adversely influence the reaction.

The reaction temperature is not critical, and the reaction can be carried out under cooling to heating.

Process 2

The object compound [Ib] or a salt thereof can be prepared by subjecting a compound [Ia] or a salt thereof to deprotection of the amino protective group.

Suitable salts of the compounds [Ia] and [Ib] may be the same as those exemplified for the compound [I].

Process 3

The object compound [Id] or a salt thereof can be prepared by subjecting a compound of [Ic] or a salt thereof to deprotection of the hydroxy protective group and the amino protective group.

Suitable method of the deprotection may include conventional one such as hydrolysis, reduction and the like.

The deprotection is typically carried out according to a similar manner to the one disclosed in Example 3(1).

Suitable salts of the compounds [Ic] and [Id] may be the same as those exemplified for the compound [I].

Process 4

The object compound [Ih] or a salt thereof can be prepared by subjecting a compound of [Ie] or a salt thereof to reduction reaction, and then by reacting the compound thus obtained or a salt thereof with a compound of [Ig] or a salt thereof.

Reduction is carried out in a conventional manner, including chemical reduction and catalytic reduction. The reduction reaction is typically carried out according to a similar manner to the one disclosed in Preparation 7.

Suitable salts of the compounds [Ih], [Ie], [If] and [Ig] may be the same as those exemplified for the compound [I].

The hydrolysis is preferably carried out in the presence of a base or an acid including Lewis acid.

Suitable base may include an inorganic base and an organic base such as metal oxide [e.g., sodium hydroxide, magnesium hydroxide, etc.], metal alkoxide [e.g., sodium methoxide, potassium methoxide, etc.], metal carbonate or metal bicarbonate, trialkylamine [e.g., trimethylamine, triethylamine, etc.], picoline, 1,5-diazabicyclo[4.3.0]non-5-ene, 1,4-diazabicyclo[2.2.2]octane, 1,8-diazabicyclo-[5.4.0]undec-7-ene, or the like.

Suitable acid may include an organic acid [e.g., formic acid, acetic acid, propionic acid, trichloroacetic acid, etc.] and an inorganic acid [e.g., hydrochloric acid, hydrobromic acid, sulfuric acid, hydrogen chloride, hydrogen bromide, ammonium chloride, etc.]. The protection using Lewis acid such as trihaloacetic acid [e.g., trichloroacetic acid, trifluoroacetic acid, etc.] or the like is preferably carried out in the presence of cation trapping agents [e.g., anisole, phenol, etc.].

The reaction is usually carried out in a solvent such as water, an alcohol [e.g., methanol, ethanol, etc.], methylene chloride, tetrahydrofuran, a mixture thereof or any other solvent which does not adversely affect the reaction.

A liquid base or acid can also be used as the solvent.

The reaction temperature is not critical and the reaction is usually carried out under cooling to warming.

Reduction is carried out in a conventional manner, including chemical reduction and catalytic reduction.

Suitable reducting agents to be used in chemical reduction are a combination of a metal (e.g., tin, zinc, iron, etc.) or metallic compound (e.g., chromium chloride, chromium acetate, etc.) and an organic or inorganic acid (e.g. formic acid, acetic acid, propionic acid, trifluoroacetic acid, p-toluenesulfonic acid, hydrochloric acid, hydrobromic acid, etc.).

Suitable catalysts to be used in catalytic reduction are conventional ones such as platinum catalysts (e.g., platinum plate, spongy platinum, platinum black, colloidal platinum, platinum oxide, platinum wire, etc.), palladium catalysts (e.g., spongy palladium, palladium black, palladium oxide, palladium on carbon, colloidal palladium, palladium on barium sulfate, palladium on barium carbonate, etc.), nickel catalysts (e.g., reduced nickel, nickel oxide, Raney nickel, etc.), cobalt catalysts (e.g., reduced cobalt, Raney cobalt, etc.), iron catalysts (e.g., reduced iron, Raney iron, etc.), copper catalysts (e.g., reduced copper, Raney copper, Ullman copper, etc.) and the like.

The reaction is usually carried out in a conventional solvent which does not adversely influence the reaction such as water, methanol, ethanol, propanol, dioxane, tetrahydrofuran, N,N-dimethylformamide, or a mixture thereof. Additionally, in case that the above-mentioned acids to be used in chemical reduction are in liquid, they can also be used as a solvent.

The reaction temperature of this reduction is not critical and the reaction is usually carried out under cooling to warming.

The compounds obtained by the above processes can be isolated and purified by a conventional method such as pulverization, recrystallization, column chromatography, reprecipitation, or the like, and converted to the desired salt in conventional manners, if necessary.

It is to be noted that the compound [I] and the other compounds may include one or more stereoisomers due to asymmetric carbon atoms, and all of such isomers and mixture thereof are included within the scope of this invention.

It is further to be noted that isomerization or rearrangement of the object compound [I] may occur due to the effect of the light acid, base or the like, and the compound obtained as the result of said isomerization or rearrangement is also included within the scope of the present invention.

It is also to be noted that the solvating form of the compound [I] (e.g. hydrate, etc.) and any form of the crystal of the compound [I] are included within the scope of the present invention.

The object compound [I] or a salt thereof possesses gut selective sympathomimetic, anti-ulcerous, anti-pancreatitis, lipolytic, anti-urinary incontinence and anti-pollakiuria activities, and are useful for the treatment and/or prevention of gastrointestinal disorders caused by smooth muscle contractions in human beings or animals, and more particularly for the treatment and/or prevention of spasm or hyperanakinesia in case of irritable bowel syndrome, gastritis, gastric ulcer, duodenal ulcer, enteritis, cholecystopathy, chlolangitis, urinary calculus and the like; for the treatment and/or prevention of ulcer such as gastric ulcer, duodenal ulcer, peptic ulcer, ulcer caused by non steroidal anti-inflammatory drugs, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis or the like; for the treatment and/or prevention of pancreatitis, obesity, diabetes, glycosuria, hyperlipidemia, hypertension, atherosclerosis, glaucoma, melancholia, depression, and the like; the treatment and/or prevention of a wasting condition, weight loss, emaciation or the like; for the treatment and/or prevention of diseases as the result of insulin resistance (e.g., hypertension, hyperinsulinemia, etc.); for the treatment and/or prevention of neurogenetic inflammation. Additionally, selective β₃ adrenergic receptor agonist are known to lower triglyceride and cholesterol levels and to raise high density lipoprotein levels in mammals (U.S. Pat. No. 5,451,677). Accordingly, the object compounds are useful in the treatment and/or prevention of conditions such as hyper-triglyceridaemea, hypercholesterolaemea and in lowering high density lipoprotein levels as well as in the treatment of atherosclerotic and cardiovascular diseases and related conditions.

Moreover, the object compound [I] is useful for inhibiting uterine contractions, preventing premature labor, and treating and preventing dysmenorrhea.

The object compound (1) or a pharmaceutically acceptable salt thereof can be usually administered to mammals including human being in the form of a conventional pharmaceutical composition such as capsule, micro-capsule, tablet, granule, powder, troche, syrup, aerosol, inhalation, solution, injection, suspension, emulsion, suppository or the like.

The effective ingredient may usually be administered with a unit dose of 0.01 mg/kg to 50 mg/kg, one to four times a day. However, the above dosage may be increased or decreased according to age, weight, conditions of patients or methods of administration.

In order to show the usefulness of the ethanolamine derivative in the present invention for the prophylactic and therapeutic treatment of above-mentioned diseases in a human being or an animal, the pharmacological test data of the representative compound thereof is shown in the following.

Test 1

Effect on the increase in intravesical pressure induced by carbachol in anesthetized dog

Test Compounds

(1) (S)-1-Phenoxy-3-[[(S)-1-hydroxy-3-(4-hydroxyphenyl)-2-propyl]amino]-2-propanol (compound of Example 6-(1) in this invention)

(2) (S)-4-[2-Hydroxy-3-[[2-[4-(5-carbamoyl-2-pyridyloxy)phenyl]-1,1-dimethylethyl]amino]propoxy]-carbazole hydrochloride (the objective compound of Example 87 (A) in EP 0827746 A1 and obtained according to a similar manner to that of the EP 0827746)

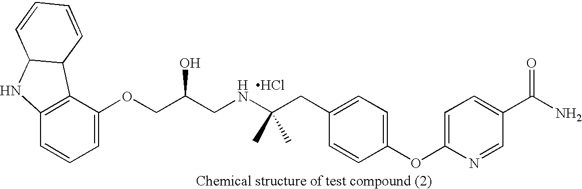

Test Method

Female Beagle dogs weighing 8.0-15.0 kg were fasted for 24 hours and maintained under halothane anesthesia. A 12F Foley catheter was lubricated with water soluble jelly, inserted into the urethral orifice and advanced approximately 10 cm until the balloon tip was placed well inside the bladder. The balloon was then inflated with 5 ml of room air and catheter slowly withdrawn just part the first resistance that is felt at the bladder neck. Urine was completely drained out through the catheter, and 30 ml of biological saline was infused. The catheter was connected to pressure transducer, and intravesical pressure was continuously recorded. The test compound was injected intravenously at five minutes before the administration of carbachol (1.8 μg/kg).

Test Results

| | Increase in intravesical |
|---|---|
| Treatment | pressure (mmHg) |
| Control | 6.8 ± 1.1 |
| Test compound (1) | 2.0 ± 0.3* |
| (0.032 mg/kg) | |
| Control | 6.9 ± 0.7 |
| Test compound (2) | 4.3 ± 0.3* |
| (0.100 mg/kg) | |

*P < 0.01 vs Control (ANOVA) (N = 3)

The above test results show that the test compounds (1) and (2) possess a relaxation effect on the smooth muscle in the urinary bladder and these compounds are useful for the treatment of pollakiuria and urinary incontinence in human beings or animals. The test compound (2) has been known as described in the above-mentioned publication. It has not been known, however, that the compound (2) is useful for the treatment of pollakiuria and urinary incontinence in human beings or animals.

The following Preparations and Examples are given for the purpose of illustrating this invention.

Preparation 1

Thionyl chloride (5.06 ml) was added dropwise to a solution of (S)-2-amino-3-(3-chloro-4-hydroxyphenyl)-propionic acid hydrochloride (5.0 g) in methanol (6 ml) under ice water cooling over 10 minutes and the mixture was stirred at room temperature for 3 hours. The mixture was evaporated in vacuo and the residue was triturated with diisopropyl ether to give (S)-2-amino-3-(3-chloro-4-hydroxyphenyl)propionic acid methyl ester hydrochloride (5.2 g). A solution of (S)-2-amino-3-(3-chloro-4-hydroxyphenyl)-propionic acid methyl ester hydrochloride (5.2 g), di-tert-butyl dicarbonate (3.75 g) and N,N-diisopropylethylamine (6.8 ml) in dioxane (50 ml) was stirred at room temperature for 3 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate and evaporated in vacuo to give (S)-2-(tert-butoxycarbonylamino)-3-(3-chloro-4-hydroxyphenyl)propionic acid methyl ester (6.44 g) as a colorless powder.

NMR (DMSO-d₆) δ: 1.30 (9H, s), 2.60-3.00 (2H, m), 3.60 (3H, s), 4.00-4.18 (1H, m), 6.83-7.20 (3H, m)

MALDI-MS (m/z): 352 (M+Na)

Preparation 2

Under nitrogen, to a solution of (S)-2-(tert-butoxycarbonylamino)-3-(3-chloro-4-hydroxyphenyl)propionic acid methyl ester (6.4 g) in methanol (30 ml) and tetrahydrofuran was added lithium borohydride (12 mg) at 5° C., and the mixture was stirred at the same temperature for 5 hours. The mixture was evaporated in vacuo. To the residue was added water and extracted with ethyl acetate. The organic layer was washed with brine, dried over sodium sulfate, and evaporated in vacuo. The residue was triturated with diisopropyl ether to give (S)-[1-(3-chloro-4-hydroxybenzyl)-2-hydroxyethyl]carbamic acid tert-butyl ester (4.63 g) as a colorless powder.

NMR (DMSO-d₆) δ: 1.30 (9H, s), 2.30-2.40 (1H, m), 2.60-2.80 (1H, m), 3.10-3.60 (3H, m), 4.68 (1H, br-s), 6.50-6.60 (1H, m), 6.80-6.95 (1H, m), 7.10-7.20 (1H, m)

MALDI-MS (m/z): 324 (M+Na)

Preparation 3

To a solution of (S)-[1-(3-chloro-4-hydroxybenzyl)-2-hydroxyethyl]carbamic acid tert-butyl ester (4.63 g) and 2,2-dimethoxy propane (8.5 ml) in dichloromethane (50 ml) was added p-toluenesulfonic acid monohydrate (12 mg) at room temperature and the mixture was stirred at the same temperature for 2 hours. The mixture was evaporated in vacuo. To the residue was added water and extracted with ethyl acetate. The organic layer was washed with brine, dried over sodium sulfate, and evaporated in vacuo. The residue was triturated with diisopropyl ether to give (S)-4-(3-chloro-4-hydroxybenzyl)-2,2-dimethyloxazolidine-3-carboxylic acid tert-butyl ester (4.44 g) as a colorless powder.

NMR (DMSO-d₆) δ: 1.30-4.50 (15H, m), 2.40-2.60 (2H, m), 3.60-4.10 (3H, m), 6.80-7.37 (3H, m)

MALDI-MS (m/z): 364 (M+Na)

Preparation 4

To a solution of (S)-4-(3-chloro-4-hydroxybenzyl)-2,2-dimethyloxazolidine-3-carboxylic acid tert-butyl ester (1.22 g) in methanol (3 ml) was added 4N hydrogen chloride in dioxane (2.0 ml) under ice water cooling, and the solution was stirred at the same temperature for 3 hours. The mixture was evaporated in vacuo and the residue was triturated with diisopropyl ether to give (S)-4-(2-amino-3-hydroxypropyl)-2-chlorophenol hydrochloride (800 mg). To a solution of (S)-4-(2-amino-3-hydroxypropyl)-2-chlorophenol hydrochloride (800 mg), acetic acid (0.2 ml) and benzaldehyde (0.34 ml) in dichloromethane (10 ml) was added sodium triacetoxyborohydride (1.07 g) at room temperature, and the mixture was stirred at the same temperature for 2 hours. The resulting mixture was poured into saturated aqueous sodium bicarbonate solution, and extracted with ethyl acetate. The organic layer was washed with brine, dried over magnesium sulfate, and evaporated in vacuo. The residue was chromatographed (hexane-ethyl acetate) over silica gel to afford (S)-4-[2-(benzylamino)-3-hydroxypropyl]-2-chlorophenol (350 mg) as a colorless powder.

NMR (CDCl₃) δ: 2.60-2.75 (2H, m), 2.80-2.90 (1H, m), 3.20-3.36 (1H, m), 3.60-3.66 (1H, m), 3.78 (2H, s), 6.90 (1H, s), 7.19-7.31 (3H, m)

MS (m/z): 292 (M+1)

Preparation 5

The following compound was obtained according to a similar manner to that of Preparation 1.

(S)-2-(tert-Butoxycarbonylamino)-3-(4-hydroxy-3-nitrophenyl)propionic acid methyl ester

NMR (DMSO-d₆) δ: 1.30 (9H, s), 2.70-3.00 (2H, m), 3.62 (3H, s), 7.05 (1H, d, J=8.5 Hz), 7.30-7.45 (2H, m), 7.70-7.80 (1H, s)

Preparation 6

The following compound was obtained according to a similar manner to that of Preparation 2.

(S)-[1-Hydroxymethyl-2-(4-hydroxy-3-nitrophenyl)-ethyl]carbamic acid tert-butyl ester

NMR (DMSO-d₆) δ: 1.27 (9H, s), 2.40-3.00 (2H, m), 3.10-3.30 (2H, m), 3.50-3.60 (1H, m), 6.50-6.55 (1H, m), 7.00-7.05 (1H, m), 7.30-7.45 (1H, m)

Preparation 7

The following compound was obtained according to a similar manner to that of Preparation 3.

(S)-4-(4-Hydroxy-3-nitrobenzyl)-2,2-dimethyl-oxazolidine-3-carboxylic acid tert-butyl ester

NMR (DMSO-d₆) δ: 1.30-1.50 (15H, m), 2.85-3.00 (2H, m), 3.85-4.10 (3H, m), 7.05 (1H, d, J=8 Hz), 7.36 (1H, d, J=8 Hz), 7.60-7.70 (1H, m)

MALDI-MS (m/z): 375 (M+Na)

Preparation 8

A mixture of (S)-4-(4-hydroxy-3-nitrobenzyl)-2,2-dimethyloxazolidine-3-carboxylic acid tert-butyl ester (10.4 g), potassium carbonate (6.1 g), benzylbromide (3.68 ml) and N,N-dimethylformamide (100 ml) was stirred at room temperature for 18 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate, and evaporated in vacuo. The residue was purified by a column chromatography on silica gel (n-hexane:ethyl acetate=3:1) to give (S)-4-[4-(benzyloxy)-3-nitrobenzyl]-2,2-dimethyloxazolidine-3-carboxylic acid tert-butyl ester (12.34 g) as a pale yellow powder.

NMR (DMSO-d₆) δ: 1.37-1.40 (15H, m), 2.80-2.90 (2H, m), 3.70-4.10 (2H, m), 5.29 (2H, s), 7.30-7.45 (7H, m), 7.60-7.69 (1H, m)

MALDI-MS (m/z): 465 (M+Na)

Preparation 9

To a solution of (S)-4-[4-(benzyloxy)-3-nitrobenzyl]-2,2-dimethyloxazolidine-3-carboxylic acid tert-butyl ester (9.36 g) in methanol (20 ml) was added 2N hydrogen chloride (100.0 ml) under ice water cooling, and the solution was stirred at the same temperature for 20 hours. The mixture was evaporated in vacuo, and the residue was triturated with diisopropyl ether to give (S)-2-amino-3-[4-(benzyloxy)-3-nitrophenyl]-1-propanol hydrochloride (8.43 g) as a pale yellow powder.

NMR (DMSO-d₆) δ: 2.90-2.95 (2H, m), 3.30-3.60 (3H, m), 5.30 (2H, s), 7.20-7.40 (6H, m), 7.50-7.55 (1H, m), 7.80-7.82 (1H, m)

MS (m/z): 303 (M+1)

Preparation 10

To a solution of (S)-2-amino-3-[4-(benzyloxy)-3-nitrophenyl]-1-propanol hydrochloride (8.3 g), acetic acid (1.4 ml) and benzaldehyde (2.49 ml) in dichloromethane (100 ml) was added sodium triacetoxyborohydride (7.8 g) at room temperature, and the mixture was stirred at the same temperature for 2 hours. The resulting mixture was poured into saturated aqueous sodium bicarbonate solution, and extracted with ethyl acetate. The organic layer was washed with brine, dried over magnesium sulfate, and evaporated in vacuo, and the residue was triturated with diisopropyl ether to give (S)-2-(benzylamino)-3-[4-(benzyloxy)-3-nitrophenyl]-1-propanol (7.62 g) as a yellow oil.

NMR (CD₃Cl) δ: 2.70-2.80 (2H, m), 2.90-2.95 (1H, m), 3.25-3.36 (1H, m), 3.60-3.65 (1H, m), 3.78 (2H, s), 5.22 (2H, s), 7.00-7.05 (1H, m), 7.20-7.50 (1H, m), 7.64-7.65 (1H, m)

MS (m/z): 393 (M+1)

Preparation 11

To a solution of (S)-[1-(hydroxymethyl)-2-(4-hydroxyphenyl)ethyl]carbamic acid tert-butyl ester (10 g) in dioxane (20 ml) was added 4N hydrogen chloride in dioxane (15 ml) at room temperature, and the solution was stirred at the same temperature for 3 hours. The mixture was evaporated in vacuo, and the residue was triturated with diisopropyl ether to give (S)-2-amino-3-(4-hydroxyphenyl)-1-propanol hydrochloride. To a solution of (S)-2-amino-3-(4-hydroxyphenyl)-1-propanol hydrochloride, acetic acid (2.14 ml) and benzaldehyde (3.8 ml) in dichloromethane (50 ml) was added sodium triacetoxyborohydride (11.9 g) at room temperature, and the mixture was stirred at the same temperature for 2 hours. The resulting mixture was poured into saturated aqueous sodium bicarbonate solution, and extracted with ethyl acetate. The organic layer was washed with brine, dried over magnesium sulfate, and evaporated in vacuo, and the residue was triturated with diisopropyl ether to give (S)-2-(benzylamino)-3-(4-hydroxyphenyl)-1-propanol (3.4 g) as a colorless powder.

NMR (CDCl₃) δ: 2.70-2.80 (2H, m), 2.85-3.00 (1H, m), 3.34 (1H, dd, J=10.7, 5.2 Hz), 3.65 (1H, dd, J=10.7, 3.8 Hz), 3.78 (2H, s), 6.70-6.80 (2H, m), 6.85-6.95 (2H, m), 7.00-7.35 (5H, m)

MS (m/z): 258 (M+1)

Preparation 12

To a mixture of (S)-2-amino-3-(4-hydroxyphenyl)-1-propanol hydrochloride (400 mg), benzaldehyde (250 mg), acetic acid (0.11 ml) and dichloromethane (10 ml), sodium triacetoxyborohydride (624 mg) and triethylamine (0.27 ml) were added. The reaction mixture was stirred at the room temperature overnight, worked up in the usual manner and purified by column chromatography (silica-gel, dichloromethane:methanol:concentrated ammonia solution=20:1:0.1) to afford (S)-2-(benzylamino)-3-(4-hydroxyphenyl)-1-propanol (202 mg).

MS (m/z): 258 (M⁺+1)

Preparation 13

To a solution of (S)-2-amino-3-phenyl-1-propanol hydrochloride, acetic acid (0.06 ml) and benzaldehyde (0.108 ml) in dichloromethane (5 ml) was added sodium triacetoxyborohydride (340 mg) at room temperature, and the mixture was stirred at the same temperature for 2 hours. The resulting mixture was poured into saturated aqueous sodium bicarbonate solution, and extracted with ethyl acetate. The organic layer was washed with brine, dried over magnesium sulfate, and evaporated in vacuo, and the residue was triturated with diisopropyl ether to give (S)-2-benzylamino-3-phenyl-1-propanol (240 mg) as a colorless powder.

NMR (CD₃Cl) δ: 2.80-2.90 (2H, m), 2.93-2.98 (1H, m), 3.30-3.50 (1H, m), 3.60-3.65 (1H, m), 3.77 (2H, s), 7.10-7.40 (10H, m)

MS (m/z): 242 (M+1)

Preparation 14

A solution of (2S)-2-(benzylamino)-3-[4-(benzyloxy)-3-nitrophenyl]-1-propanol (620 mg), di-tert-butyl dicarbonate (414 mg) and triethylamine (1 ml) in dioxane (20 ml) was stirred at room temperature for 3 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate and evaporated in vacuo. A mixture of the residue (800 mg), iron-powder (800 mg), ammonium chloride (80 mg), water (1.6 ml) and ethanol (8.0 ml) was heated under reflux for 4 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate, and evaporated in vacuo to give (2S)-2-[N-benzyl-N-(tert-butoxycarbonyl)amino]-3-[3-amino-4-(benzyloxy)phenyl]-1-propanol (680 mg) as a pale yellow foam.

MS (m/z): 463 (M+1)

Preparation 15

Benzenesulfonyl chloride (321 mg) was added to a solution of (2S)-2-[N-(benzyl-N-(tert-butoxycarbonyl)amino]-3-[3-amino-4-(benzyloxy)phenyl]-1-propanol (680 mg) and pyridine (0.1 ml) in dichloromethane (5 ml) under ice water cooling over 10 minutes and the mixture was stirred at room temperature for a further 1 hour. To this one was added aqueous saturated solution of sodium bicarbonate (5.0 ml). The mixture was stirred at the same temperature for 18 hours, and which was dissolved in ethyl acetate, washed with aqueous saturated sodium bicarbonate solution and brine, dried over sodium sulfate, and evaporated in vacuo. To a solution of the residue (700 mg) in dioxane (20 ml) was added 4N hydrogen chloride in dioxane (5 ml) at room temperature, and the solution was stirred at the same temperature for 3 hours. The mixture was dissolved in ethyl acetate, washed with aqueous saturated sodium bicarbonate solution and brine, dried over sodium sulfate, and evaporated in vacuo to give N-[5-[(2S)-2-(benzylamino)-3-hydroxypropyl]-2-(benzyloxy)phenyl]-benzenesulfonamide (510 mg) as a yellow oil.

MS (m/z): 503 (M+1)

Preparation 16

The following compound was obtained according to a similar manner to that of Preparation 1.

(2S)-2-[(tert-Butoxycarbonyl)amino]-3-(3-fluoro-4-hydroxyphenyl)propionic acid methyl ester

NMR (DMSO-d₆) δ: 1.32 (9H, s), 2.60-3.00 (2H, m), 3.60 (3H, s), 4.00-4.20 (1H, m), 6.80-7.10 (3H, m), 7.20-7.30 (1H, m)

MS (m/z): 336 (M+Na)

Preparation 17

The following compound was obtained according to a similar manner to that of Preparation 2.

(1S)-[1-(3-Fluoro-4-hydroxybenzyl)-2-hydroxyethyl]carbamic acid tert-butyl ester

NMR (DMSO-d₆) δ: 1.32 (9H, s), 2.40-2.70 (2H, m), 3.20-3.30 (2H, m), 3.40-3.60 (1H, m), 6.50-6.90 (4H, m)

MS (m/z): 308 (M+Na)

### EXAMPLE 1

A mixture of (S)-1-[4-(benzyloxy)-3-(hydroxymethyl)-phenoxy]-3-[N-benzyl-N-[(S)-1-hydroxy-3-(4-hydroxyphenyl)-2-propyl]amino]-2-propanol (150 mg), 10% palladium on activated carbon (50% wet, 50 mg) and methanol (5 ml) was stirred at room temperature in the presence of hydrogen at an atmospheric pressure for 1 hour, and filtered. The filtrate was evaporated in vacuo. The residue was chromatographed (chloroform-methanol) over silica gel to afford (S)-1-[4-hydroxy-3-(hydroxymethyl)phenoxy]-3-[[(S)-1-hydroxy-3-(4-hydroxyphenyl)-2-propyl]amino]-2-propanol (65 mg) as a colorless foam.

IR (KBr): 3380-3200, 1612, 1598, 1511, 1444, 1205 cm⁻¹

NMR (CD₃OD) δ: 2.51-3.00 (5H, m), 3.32-3.65 (2H, m), 3.86 (2H, d, J=5.2 Hz), 3.90-4.00 (1H, m), 4.62 (2H, s), 6.60-6.80 (4H, m), 6.90 (1H, s), 7.03 (2H, d, J=8.4 Hz)

MS (m/z): 364 (M+1)

### EXAMPLE 2

4-Chlorobenzenesulfonyl chloride (23.9 mg) was added to a solution of (S)-1-[3-amino-4-(benzyloxy)phenoxy]-3-[N-benzyl-N-[(S)-1-hydroxy-3-[4-(benzyloxy)phenyl]-2-propyl]amino]-2-propanol (110 mg) and pyridine (0.1 ml) in dichloromethane (6 ml) under ice water cooling over 10 minutes and the mixture was stirred at room temperature for a further 1 hour. To this one was added aqueous saturated solution of sodium bicarbonate (5.0 ml). The mixture was stirred at the same temperature for 18 hours, and which was dissolved in ethyl acetate, washed with aqueous saturated sodium bicarbonate solution and brine, dried over sodium sulfate, and evaporated in vacuo. A mixture of the residue and 10% palladium on activated carbon (50% wet, 10 mg) in methanol (2.0 ml) and chlorobenzene (2.0 ml) was stirred at room temperature in the presence of hydrogen at an atmospheric pressure for 1 hour, and filtered. The filtrate was evaporated in vacuo. The residue was chromatographed (chloroform-methanol) over silica gel to afford (S)-1-[3-[(4-chlorobenzenesulfonyl)amino]-4-hydroxyphenoxy]-3-[[(S)-1-hydroxy-3-(4-hydroxyphenyl)-2-propyl]amino]-2-propanol (15 mg) as a brown foam.

IR (KBr): 3345-3000, 1612, 1589, 1515, 1328, 1160 cm⁻¹

NMR (CD₃OD) δ: 2.50-3.05 (5H, m), 3.32-3.85 (2H, m), 3.82 (2H, d, J=5.0 Hz), 4.00-4.10 (1H, m), 6.50-6.80 (4H, m), 7.01-7.08 (3H, m), 7.30-7.35 (2H, m), 7.40-7.50 (2H, m)

MALDI-MS (m/z): 523 (M⁺)

### EXAMPLE 3

The following compounds were obtained according to a similar manner to that of Example 2.

(1) (R)-1-[3-(Benzenesulfonylamino)-4-hydroxyphenyl]-2-[[(S)-1-hydroxy-3-(3-chloro-4-hydroxyphenyl)-2-propyl]amino]ethanol

IR (KBr): 3300-3000, 1612, 1589, 1479, 1288 cm⁻¹

NMR (CD₃OD) δ: 2.60-2.80 (4H, m), 3.32-3.50 (3H, m), 4.54 (2H, t, J=6.1 Hz), 6.60-6.90 (4H, m), 7.09-7.55 (5H, m), 7.70-7.90 (2H, m)

MALDI-MS (m/z): 493 (M+1)

(2) (S)-1-Phenoxy-3-[[(S)-3-[3-(benzenesulfonylamino)-4-hydroxyphenyl]-1-hydroxy-2-propyl]amino]-2-propanol

IR (KBr): 3500-3000, 1596, 1496, 1326, 1162 cm⁻¹

NMR (CD₃OD) δ: 2.40-3.05 (5H, m), 3.20-3.60 (2H, m), 3.91 (2H, d, J=5.2 Hz), 4.00-4.05 (1H, m), 6.60 (1H, d, J=8.3 Hz), 6.70-6.95 (4H, m), 7.10-7.30 (6H, m), 7.75 (2H, d, J=8.3 Hz)

MS (m/z): 473 (M+1)

### EXAMPLE 4

Under nitrogen, a solution of (S)-4-(2-amino-3-hydroxypropyl)phenol hydrochloride (600 mg), (2S)-1,2-epoxy-3-phenoxypropane (308 mg) and N,N-diisopropylethylamine (0.51 ml) in ethanol (10 ml) was refluxed for 3 hours. The mixture was evaporated in vacuo. The residue was chromatographed (chloroform-methanol) over silica gel to afford (S)-1-phenoxy-3-[[(S)-1-hydroxy-3-(4-hydroxyphenyl)-2-propyl]amino]-2-propanol. To a solution of (S)-1-phenoxy-3-[[(S)-1-hydroxy-3-(4-hydroxyphenyl)-2-propyl]amino]-2-propanol in dioxane (2 ml) was added 4N hydrogen chloride in dioxane (2 ml) at room temperature, and the solution was stirred at the same temperature for 4 hours. The mixture was evaporated in vacuo, and the residue was triturated with diisopropyl ether to give (S)-1-phenoxy-3-[[(S)-1-hydroxy-3-(4-hydroxyphenyl)-2-propyl]amino]-2-propanol hydrochloride (60 mg) as a colorless powder.

IR (KBr): 3315-3200, 1594, 1513, 1455, 1240 cm⁻¹

NMR (D₃O) δ: 2.90-3.03 (2H, m), 3.25-3.40 (2H, m), 3.60-4.00 (3H, m), 4.10-4.20 (2H, m), 4.30-4.40 (1H, m), 6.80-7.35 (7H, m), 7.40-7.53 (2H, m)

MS (m/z): 318 (M⁺)

### EXAMPLE 5

A mixture of (S)-4-(oxiranylmethoxy)-1H-indole (38.3 mg), (S)-2-amino-3-(4-hydroxyphenyl)-1-propanol hydrochloride (44 mg), methanol (2 ml) and N,N-diisopropylethylamine (58 μl) was heated under reflux for 2 hours, evaporated and purified by preparative thin-layer chromatography (dichloromethane:methanol:concentrated ammonia solution=7:1:0.1) to afford (S)-1-(1H-indol-4-yloxy)-3-[[(S)-1-hydroxy-3-(4-hydroxyphenyl)-2-propyl]amino]-2-propanol (46.1 mg).

IR (KBr): 1512 (m), 1362 (m), 1244 (s), 750 (m) cm⁻¹

NMR (CD₃OD) δ: 2.61-3.17 (5H, m), 3.45 (1H, dd, J=6.1, 11.4 Hz), 3.63 (1H, dd, J=3.9, 11.5 Hz), 4.0-4.3 (3H, m), 6.5-6.6 (2H, m), 6.70 (2H, d, J=8.4 Hz), 7.0-7.2 (5H, m)

MS (m/z): 357 (M⁺+1)

### EXAMPLE 6

The following compounds were obtained according to a similar manner to that of Example 5.

(1) (S)-1-Phenoxy-3-[[(S)-1-hydroxy-3-(4-hydroxyphenyl)-2-propyl]amino]-2-propanol

IR (KBr): 1597 (m), 1454 (m), 1246 (s), 1038 (s), 854 (m) cm⁻¹

NMR (CD₃OD) δ: 2.49-2.96 (5H, m), 3.37 (1H, dd, J=6.4, 11.0 Hz), 2.55 (1H, dd, J=4.2, 11.0 Hz), 3.9-4.1 (3H, m), 6.69 (2H, d, J=8.5 Hz), 6.9-7.0 (3H, m), 7.02 (2H, d, J=8.4 Hz), 7.23 (1H, d, J=8.1 Hz), 7.27 (1H, d, J=7.9 Hz)

MS (m/z): 318 (M⁺+1)

(2) (S)-1-(3-Pyridyloxy)-3-[[(S)-1-hydroxy-3-(4-hydroxyphenyl)-2-propyl]amino]-2-propanol

IR (KBr): 1514 (m), 1275 (s), 1238 (s), 1111 (m), 804 (m) cm⁻¹

NMR (CD₃OD) δ: 2.5-3.0 (5H, m), 3.39 (1H, dd, J=6.4, 11.0 Hz), 3.56 (1H, dd, J=4.2, 11.1 Hz), 3.9-4.1 (3H, m), 6.69 (2H, d, J=8.4 Hz), 7.03 (2H, d, J=8.4 Hz), 7.3-7.5 (2H, m), 8.12 (1H, d, J=2.8 Hz), 8.24 (1H, d, J=2.5 Hz)

MS (m/z): 319 (M⁺+1)

### EXAMPLE 7

A mixture of (S)-1-[4-(benzyloxy)-3-nitrophenoxy]-3-[N-benzyl-N-[(S)-1-hydroxy-3-[4-(benzyloxy)phenyl]-2-propyl]amino]-2-propanol (85 mg), iron powder (0.1 g), ammonium chloride (0.01 g), ethanol (1 ml) and water (0.1 ml) was heated under reflux for 1 hour. The reaction mixture was filtrated and worked up in the usual manner to give (S)-1-(3-amino-4-nitrophenoxy)-3-[N-benzyl-N-[(S)-1-hydroxy-3-[4-(benzyloxy)phenyl]-2-propyl]amino]-2-propanol as a crude product. To the crude product, dichloromethane (1 ml), pyridine (64 μl) and benzenesulfonyl chloride (52 mg) were added. After the reaction mixture was stirred at the room temperature for 0.5 hours, ethyl acetate (10 ml) and saturated aqueous sodium bicarbonate solution (10 ml) were added, stirred at the room temperature overnight and worked up in the usual manner to give (S)-1-[3-(benzenesulfonylamino)-4-nitrophenoxy]-3-[N-benzyl-N-[(S)-1-hydroxy-3-[4-(benzyloxy)phenyl]-2-propyl]amino]-2-propanol as a crude product. The crude product was hydrogenated by using palladium on charcoal in the usual manner and purified by preparative thin-layer chromatography (dichloromethane:methanol:concentrated ammonia solution=5:1:0.1) to afford (S)-1-[3-(benzenesulfonylamino)-4-hydroxyphenoxy]-3-[[(S)-1-hydroxy-3-(4-hydroxyphenyl)-2-propyl]amino]-2-propanol (38 mg).

IR (KBr): 1616 (m), 1514 (s), 1456 (m), 1163 (s) cm⁻¹

NMR (CD₃OD) δ: 2.5-3.0 (5H, m), 3.33 (1H, dd, J=6.4, 11.0 Hz), 3.56 (1H, dd, J=4.2, 11.0 Hz), 3.7-3.8 (2H, m), 3.9-4.0 (1H, m), 6.49 (1H, dd, J=2.8, 8.8 Hz), 6.59 (1H, d, J=8.8 Hz), 6.70 (2H, d, J=8.4 Hz), 6.92 (1H, d, J=2.8 Hz), 7.03 (2H, d, J=8.5 Hz), 7.4-7.6 (3H, m), 7.75-7.79 (2H, m)

MS (m/z): 489 (M⁺+1)

### EXAMPLE 8

The following compound was obtained according to a similar manner to that of Example 7.

(R)-1-[3-(Benzenesulfonylamino)-4-hydroxyphenyl]-2-[[(S)-1-hydroxy-3-(4-hydroxyphenyl)-2-propyl]amino]ethanol

IR (KBr): 1649 (s), 1514 (s), 1163 (m), 822 (m) cm⁻¹

NMR (CD₃OD) δ: 2.5-2.9 (5H, m), 3.38 (1H, dd, J=6.4, 11.0 Hz), 3.6 (1H, dd, J=4.2, 11.0 Hz), 4.55 (1H, t, J=7 Hz), 6.6-6.8 (3H, m), 6.8-7.1 (3H, m), 7.27 (1H, s), 7.3-7.6 (3H, m), 7.7-7.8 (2H, m)

MS (m/z): 459 (M⁺+1)

### EXAMPLE 9

Under nitrogen, a solution of (S)-4-(2-benzylamino-3-hydroxypropyl)phenol (150 mg) and (S)-2-(4-benzyloxy-3-formyl)phenoxymethyloxirane (166 mg) in ethanol (5 ml) was refluxed for 24 hours. The mixture was evaporated in vacuo. The residue was purified by a column chromatography on silica gel (hexane:ethyl acetate=1:1), to give 4-[(2S)-2-[N-benzyl-N-[(2S)-3-(4-benzyloxy-3-formylphenoxy)-2-hydroxypropyl]amino]-3-hydroxypropyl]phenol (170 mg). Under nitrogen, to a solution of 4-[(2S)-2-[N-benzyl-N-[(2S)-3-(4-benzyloxy-3-formylphenoxy)-2-hydroxypropyl]amino]-3-hydroxypropyl]phenol (170 mg) in methanol (3 ml) was added sodium borohydride (12 mg) at 5° C., and the mixture was stirred at the same temperature for 30 minutes. The mixture was evaporated in vacuo. To the residue was partitioned between water and ethyl acetate. The organic layer was washed with brine, dried over sodium sulfate, and evaporated in vacuo. The residue was purified by a column chromatography on silica gel (chloroform:methanol=100:1) to give (S)-1-[4-benzyloxy-3-(hydroxymethyl)phenoxy]-3-[N-benzyl-N-[(S)-1-hydroxy-3-(4-hydroxyphenyl)-2-propyl]amino]-2-propanol (150 mg) as a colorless foam.

NMR (CD₃Cl) δ: 2.40-2.60 (2H, m), 2.65-3.00 (3H, m), 3.05-3.10 (1H, m), 3.40-3.95 (8H, m), 4.68 (2H, s), 5.04 (2H, s), 6.60-7.00 (8H, m), 7.20-7.40 (6H, m)

MS (m/z): 544 (M+1)

### EXAMPLE 10

A mixture of (S)-1-[3-nitro-4-(benzyloxy)phenoxy]-3-[N-benzyl-N-[(S)-1-hydroxy-3-[4-(benzyloxy)phenyl]-2-propyl]amino]-2-propanol (178 mg), iron-powder (178 mg), ammonium chloride (17.8 mg), water (0.3 ml) and ethanol (10 ml) was heated under reflux for 6 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate, and evaporated in vacuo to give (S)-1-[3-amino-4-(benzyloxy)phenoxy]-3-[N-benzyl-N-[(S)-1-hydroxy-3-[4-(benzyloxy)phenyl]-2-propyl]amino]-2-propanol (110 mg) as a pale yellow foam.

MS (m/z): 619 (M+1)

### EXAMPLE 11

The following compound was obtained according to a similar manner to that of Example 10.

(R)-1-[3-Amino-4-(benzyloxy)phenyl]-2-[N-benzyl-N-[(S)-1-hydroxy-3-[4-(benzyloxy)-3-chlorophenyl]-2-propyl]amino]-ethanol

NMR (CDCl₃) δ: 2.40-2.90 (4H, m), 2.95-3.05 (1H, m), 3.40-3.99 (5H, m), 4.40-4.48 (1H, m), 5.05 (2H, s), 5.13 (2H, s), 6.50-7.47 (21H, m)

MS (m/z): 623 (M⁺)

### EXAMPLE 12

Under nitrogen, a solution of (S)-2-(benzylamino)-3-[4-(benzyloxy)-3-nitrophenyl]-1-propanol (3.0 g) and (2S)-1,2-epoxy-3-phenoxypropane (2.45 g) in ethanol (50 ml) was refluxed for 24 hours. The mixture was evaporated in vacuo. The residue was purified by a column chromatography on silica gel (hexane:ethyl acetate=1:1) to give (S)-1-phenoxy-3-[N-benzyl-N-[(S)-3-[4-(benzyloxy)-3-nitrophenyl]-1-hydroxy-2-propyl]amino]-2-propanol (2.49 g). A mixture of (S)-1-phenoxy-3-[N-benzyl-N-[(S)-3-[4-(benzyloxy)-3-nitrophenyl]-1-hydroxy-2-propyl]amino]-2-propanol (2.49 g), iron-powder (2.49 g), ammonium chloride (0.25 g), water (5 ml) and ethanol (25 ml) was refluxed for 2 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate, and evaporated in vacuo to give (S)-1-phenoxy-3-[N-benzyl-N-[(S)-3-[3-amino-4-(benzyloxy)phenyl]-1-hydroxy-2-propyl]amino]-2-propanol (2.24 g) as a brown foam.

NMR (DMSO-d₆) δ: 2.20-2.40 (1H, m), 2.60-3.90 (4H, m), 3.40-3.55 (2H, m), 3.70-4.10 (3H, m), 4.28 (1H, br-s), 4.63 (1H, s), 4.73 (1H, br-s), 5.02 (1H, s), 6.32 (1H, d, J=8.4 Hz), 6.47 (1H, s), 6.70-6.90 (2H, m), 7.10-7.50 (12H, m)

MS (m/z): 513 (M+1)

### EXAMPLE 13

A mixture of (S)-2-[[4-(benzyloxy)-3-nitrophenoxy]-methyl]oxirane (56 mg), (S)-2-(benzylamino)-3-(4-hydroxyphenyl)-1-propanol (48 mg) and ethanol (2 ml) was heated under reflux for 20 hours and evaporated to give (S)-1-[4-(benzyloxy)-3-nitrophenoxy]-3-[[(S)-1-hydroxy-3-[4-(benzyloxy)phenyl]-2-propyl]amino]-2-propanol as a crude residue. To the crude residue, benzyl bromide (35 mg), potassium carbonate (57 mg) and N,N-dimethylformamide (1 ml) was added. The mixture was heated at 60° C. for 3.5 hours, worked up in the usual manner and purified by preparative thin-layer chromatography (hexane:ethyl acetate=1.2:1) to afford (S)-1-[4-(benzyloxy)-3-nitrophenoxy]-3-[N-benzyl-N-[(S)-1-hydroxy-3-[4-(benzyloxy)phenyl]-2-propyl]amino]-2-propanol (89 mg).

MS (m/z): 649 (M⁺+1)

### EXAMPLE 14

The following compounds were obtained according to a similar manner to that of Example 13.

(1) (R)-1-[4-(Benzyloxy)-3-nitrophenyl]-2-[N-benzyl-N-[(S)-1-hydroxy-3-[4-(benzyloxy)-3-chlorophenyl]-2-propyl]amino]ethanol

NMR (CDCl₃) δ: 2.50-2.95 (4H, m), 3.00-3.05 (1H, m), 3.40-3.60 (2H, m), 4.40-4.50 (1H, m), 5.13 (2H, s), 5.20 (2H, s), 6.80-7.40 (20H, m), 7.65-7.70 (1H, m)

MS (m/z): 653 (M⁺)

(2) (R)-1-[4-(Benzyloxy)-3-nitrophenyl]-2-[N-benzyl-N-[(S)-1-hydroxy-3-[4-(benzyloxy)phenyl]-2-propyl]amino]ethanol

MS (m/z): 619 (M⁺+1)

### EXAMPLE 15

To a solution of (S)-2-[4-(methoxymethoxy)phenyl]-1-(methoxymethyl)ethylamine (108 mg) in ethanol (4.8 ml) was added (S)-3-phenoxy-1,2-epoxypropane (86.3 mg) and the solution was refluxed for 7.5 hours. After cooling to room temperature, the solvent was removed by evaporation and the residue was chromatographed on a 10 g of silica gel (eluent: hexane/ethyl acetate=2/1 to 1/1, then ethyl acetate only) to give (2S)-1-[(1S)-2-[4-(methoxymethoxy)phenyl]-1-(methoxymethyl)ethylamino]-3-(phenoxy)propan-2-ol (106 mg) as a pale yellow oil.

IR (Neat): 3423, 2925, 1598, 1244, 1151, 1078, 1007 cm⁻¹

NMR (CDCl₃) δ: 1.75 (2H, br), 2.68-2.95 (5H, m), 3.22-3.39 (2H, m), 3.34 (3H, s), 3.48 (3H, s), 3.91-3.97 (3H, m), 5.15 (2H, s), 6.88-6.99 (3H, m), 7.08-7.13 (2H, m), 7.24-7.32 (2H, m)

MS (m/z): 376 (MH⁺)

### EXAMPLE 16

To a solution of (2S)-1-[(1S)-2-[4-(methoxymethoxy)-phenyl]-1-(methoxymethyl)ethylamino]-3-(phenoxy) propan-2-ol (91.6 mg) in a mixed solvent of dioxane (1.0 ml) and methanol (1.0 ml) was added 4N hydrogen chloride in dioxane (2.0 ml) and the solution was stirred at the same temperature for 1 hour. The solvent was evaporated in vacuo to give 4-[(2S)-2-[(2S)-2-hydroxy-3-(phenoxy)propylamino]-3-(methoxy)propyl]phenol hydrochloride (88.1 mg) as a pale yellow solid.

IR (KBr): 3380 (br), 1595, 1516, 1242 cm⁻¹

NMR (DMSO-d₆) δ: 2.66-3.21 (5H, m), 3.26 (3H, s), 3.43-3.51 (2H, m), 5.22 (2H, d, J=4.0 Hz), 4.23 (1H, br), 5.87 (1H, brd, J=4.8 Hz), 6.73 (2H, d, J=8.3 Hz), 6.93-6.99 (3H, m), 7.05-7.35 (2H, m), 8.60 (1H, br), 8.96 (1H, br), 9.38 (1H, s)

MS (m/z): 332 (M—Cl⁻)

### EXAMPLE 17

To a solution of (S)-3-amino-4-[4-(methoxymethoxy)-phenyl]-2-methylbutan-2-ol (542 mg) in ethanol (23 ml) was added (S)-3-phenoxy-1,2-epoxypropane (407 mg) and the solution was refluxed for 8 hours. The solvent was evaporated and the residue was chromatographed on a 50 g of silica gel (eluent: chloroform/methanol=95/5) to give (3S)-3-[((2S)-2-hydroxy-3-phenoxypropyl)amino]-4-[4-(methoxymethoxy)phenyl]-2-methylbutan-2-ol (339 mg) as a pale yellow oil.

MS (m/z): 390 (MH⁺)

### EXAMPLE 18

To a solution of (3S)-3-[((2S)-2-hydroxy-3-phenoxypropyl)amino]-4-[4-(methoxymethoxy)phenyl]-2-methyl-butan-2-ol (100 mg) in a mixed solvent of dioxane (1.0 ml) and methanol (1.0 ml) was added 4N hydrogen chloride in dioxane (1.0 ml) and the solution was stirred at the room temperature for 1 hour. The solvent was removed by evaporation to give 4-[(2S)-3-hydroxy-2-[((2S)-2-hydroxy-3-phenoxypropyl)amino]-3-methylbutyl]phenol hydrochloride (99.9 mg) as a pale yellow solid.

IR (KBr): 3423, 1597, 1516, 1240 cm⁻¹

NMR (DMSO-d₆) δ: 1.19 (3H, s), 1.23 (3H, s), 2.63-3.26 (5H, m), 3.76 (1H, dd, J=5.9, 10.0 Hz), 3.91 (1H, dd, J=5.1, 10.0 Hz), 4.14 (1H, br), 5.83 (1H, br), 6.05 (1H, d, J=5.0 Hz), 6.73 (2H, d, J=8.4 Hz), 6.86-6.98 (3H, m), 7.19 (2H, d, J=8.4 Hz), 7.25-7.33 (2H, m), 9.40 (1H, br-s), 9.64 (1H, br)

MS (m/z): 346 (M—Cl⁻)

### EXAMPLE 19

Under nitrogen, a solution of (S)-2-benzylamino-3-phenyl-1-propanol (1.0 g), (S)-[[2-(benzyloxy)-3-nitrophenoxy]methyl]oxirane (1.25 g) in ethanol (10 ml) was refluxed for 24 hours. The mixture was evaporated in vacuo. The residue was purified by a column chromatography on silica gel (hexane:ethyl acetate=1:1) to give (2S)-2-[N-benzyl-N-[(2S)-3-[4-(benzyloxy)-3-nitrophenoxy]-2-hydroxypropyl]amino]-3-phenyl-1-propanol (1.90 g) as a yellow foam.

NMR (CHCl₃) δ: 2.40-2.90 (4H, m), 3.10-3.20 (1H, m), 3.50-3.95 (6H, m), 5.18 (1H, s), 7.00-7.50 (15H, m)

MS (m/z): 543 (M+1)

### EXAMPLE 20

The following compounds were obtained according to a similar manner to that of Example 10.

(1) (S)-1-[3-Amino-4-(benzyloxy)phenoxy]-3-[N-benzyl-N-(S)-1-hydroxy-3-phenyl-2-propyl) amino]-2-propanol

NMR (CHCl₃) δ: 2.50 (1H, q, J=8.8, 13.4 Hz), 2.70-3.20 (3H, m), 3.40-3.90 (8H, m), 5.00 (2H, s), 6.10-6.15 (2H, m), 6.70 (1H, m), 7.10-7.44 (15H, m)

MS (m/z): 513 (M+1)

(2) (R)-1-[3-Amino-4-(benzyloxy)phenyl]-2-[N-benzyl-N-((S)-1-hydroxy-3-phenyl-2-propyl)amino]ethanol

MS (m/z): 483 (M+1)

(3) ((2S)-2-[N-Benzyl-N-[(2R)-2-[3-amino-4-(benzyloxy)phenyl]-2-hydroxyethyl]amino]-3-(4-methoxyphenyl)-1-propanol

MS (m/z): 513 (M+1)

(4) (2S)-2-[N-Benzyl-N-[(2S)-3-[3-amino-4-(benzyloxy)phenoxy]-2-hydroxypropyl]amino]-3-(4-methoxyphenyl)-1-propanol

MS (m/z): 543 (M+1)

(5) (2S)-2-[N-Benzyl-N-[(2S)-3-[3-amino-4-(benzyloxy)phenoxy]-2-hydroxypropyl]amino]-3-[3-chloro-4-(benzyloxy)phenyl]-1-propanol

MS (m/z): 653 (M+)

### EXAMPLE 21

4-Fluorobenzenesulfonyl chloride (40.7 mg) was added to a solution of (S)-1-[3-amino-4-(benzyloxy)phenoxy]-3-[N-benzyl-N-((S)-1-hydroxy-3-phenyl-2-propyl)amino]-2-propanol (107 mg) and pyridine (0.1 ml) in dichloromethane (5 ml) under ice water cooling over 10 minutes and the mixture was stirred at room temperature for a further 1 hour. To this one was added aqueous saturated solution of sodium bicarbonate (5.0 ml). The mixture was stirred at the same temperature for 18 hours, and which was dissolved in ethyl acetate, washed with aqueous saturated sodium bicarbonate solution and brine, dried over sodium sulfate, and evaporated in vacuo. A mixture of the residue, 10% palladium on activated carbon (50% wet, 30 mg), methanol (5.0 ml) and chlorobenzene (5.0 ml) was stirred at room temperature in the presence of hydrogen at an atmospheric pressure for 1 hour, and filtered. The filtrate was evaporated in vacuo. The residue was chromatographed (chloroform-methanol) over silica gel to afford (S)-1-[3-[(4-fluorobenzenesulfonyl)amino]-4-hydroxyphenoxy]-3-[[(S)-1-hydroxy-3-phenyl-2-propyl]amino]-2-propanol (50 mg) as a brown foam.

IR (KBr): 3380-3000, 1592, 1496, 1407, 1328, 1153, 1039 cm⁻¹

NMR (CD₃OD) δ: 2.60-3.00 (5H, m), 3.30-3.40 (2H, m), 3.80 (2H, d, J=4.2 Hz), 4.05-4.10 (1H, m), 6.45-6.60 (2H, m), 7.00-7.33 (8H, m), 7.70-7.80 (2H, m)

MS (m/z): 491 (M+1)

### EXAMPLE 22

The following compounds were obtained according to a similar manner to that of Example 21.

(1) (S)-1-[3-[(4-Chlorobenzenesulfonyl)amino]-4-hydroxyphenoxy]-3-[[(S)-1-hydroxy-3-phenyl-2-propyl]amino]-2-propanol

IR (KBr): 3380−3000, 1612, 1589, 1328, 1159, 1045 cm⁻¹

NMR (CD₃OD) δ: 2.80-3.80 (7H, m), 3.90-4.00 (2H, m), 4.20-4.30 (1H, m), 6.50-6.60 (2H, m), 7.00 (1H, d, J=2.5 Hz), 7.20-7.40 (7H, m), 7.70-7.80 (2H, m)

(2) N-[5-[(1R)-2-[((1S)-1-benzyl-2-hydroxyethyl)amino]-1-hydroxyethyl]-2-hydroxyphenyl]-benzenesulfonamide

IR (KBr): 3380-3000, 1604, 1494, 1446, 1162, 1089, 1153, 1087 cm⁻¹

NMR (CD₃OD) δ: 2.60-3.10 (5H, m), 3.40-3.70 (2H, m), 4.50 (1H, t, J=6.20 Hz), 6.60 (1H, d, J=8.2 Hz), 6.90 (1H, d, J=8.2 Hz), 7.10-7.40 (9H, m), 7.70-7.80 (2H, m)

MS (m/z): 443 (M+1)

(3) N-[5-[(1R)-2-[((1S)-1-Benzyl-2-hydroxyethyl)amino]-1-hydroxyethyl]-2-hydroxyphenyl]-4-fluorobenzenesulfonamide

IR (KBr): 3380-3000, 1592, 1494, 1407, 1328, 1168, 1153, 1087 cm⁻¹

NMR (CD₃OD) δ: 2.60-3.00 (4H, m), 3.40-3.60 (3H, m), 4.60 (1H, t, J=5.9 Hz), 6.60 (1H, d, J=8.3 Hz), 6.90 (1H, d, J=8.3 Hz), 7.05-7.38 (8H, m), 7.70-7.80 (2H, m)

MS (m/z): 461 (M+1)

(4) N-[5-[(1R)-2-[((1S)-1-Benzyl-2-hydroxyethyl)amino]-1-hydroxyethyl]-2-hydroxyphenyl]-propanesulfonamide

IR (KBr): 3380-3000, 1602, 1446, 1326, 1290, 1110 cm⁻¹

NMR (CD₃OD δ: 1.00 (3H, t, J=8.0 Hz), 1.80 (2H, q, J=8.0 Hz), 2.70-3.10 (7H, m), 3.40-3.70 (2H,m), 4.61 (1H, t, J=6.20 Hz), 6.70-6.80 (1H, m), 7.00-7.05 (1H, m), 7.10-7.40 (6H, m)

MS (m/z): 423 (M+1)

(5) N-[5-[(1R)-2-[((1S)-1-Benzyl-2-hydroxyethyl)amino]-1-hydroxyethyl]-2-hydroxyphenyl]-butanesulfonamide

IR (KBr): 3380-3000, 1612, 1407, 1326, 1292, 1145, 1110 cm⁻¹

NMR (CD₃OD) δ: 0.90 (3H, t, J=7.3 Hz), 1.30-1.50 (2H, m), 1.70-1.90 (2H, m), 2.70-3.10 (7H, m), 3.40-3.60 (2H, m), 6.80 (lH, d, J=8.2 Hz), 7.00 (1H, d, J=8.2 Hz), 7.10-7.40 (6H, m)

MS (m/z): 409 (M+1)

(6) (S)-1-[3-(benzenesulfonylamino)-4-hydroxyphenoxy]-3-[((S)-1-hydroxy-3-phenyl-2-propyl)amino]-2-propanol

IR (KBr): 3380-3050, 1604, 1446, 1326, 1159, 1089, 1043 cm⁻¹

NMR (CD₃OD) δ: 2.60-3.00 (5H, m), 3.40-3.70 (2H, m), 3.80 (2H, d, J=5.2 Hz), 3.90-3.95 (1H, m), 6.45-6.60 (2H, m), 6.90 (1H, d, J=2.7 Hz), 7.10-7.70 (8H, m), 7.70-7.80 (2H, m)

MS (m/z): 473 (M+1)

(7) N-[2-Hydroxy-5-[(1R)-1-hydroxy-2-[[(1S)-2-hydroxy-1-(4-methoxybenzyl)ethyl]amino]ethyl]phenyl]-benzenesulfonamide

IR (KBr): 3380-3000, 1610, 1513, 1448, 1324, 1245, 1162 cm⁻¹

NMR (CD₃OD) δ: 2.60-2.95 (4H, m), 3.30-3.60 (4H, m), 3.74 (3H, s), 4.56 (1H, t, J=6.5 Hz), 6.60-7.00 (2H, m), 7.05-7.25 (2H, m), 7.30-7.60 (4H, m), 7.70-7.80 (2H, m)

MS (m/z): 473 (M+1)

(8) (S)-1-[3-(Benzenesulfonylamino)-4-hydroxyphenoxy]-3-[[(S)-1-hydroxy-3-(4-methoxyphenyl)-2-propyl]amino]-2-propanol

IR (KBr): 3400-3000, 1610, 1515, 1446, 1328, 1247, 1168, 1157, 1087 cm⁻¹

NMR (CD₃OD) δ: 2.50-3.00 (4H, m), 3.30-3.60 (3H, m), 3.66 (3H, s), 3.80 (2H, d, J=4.6 Hz), 6.40-6.70 (2H, m), 6.80 (2H, d, J=8.5 Hz), 7.00 (1H, d, J=2.6 Hz), 7.10 (2H, d, J=8.5 Hz), 7.30-7.60 (3H, m), 7.70-7.80 (2H, m)

MS (m/z): 503 (M+1)

(9) (S)-1-[3-(4-Fluorobenzenesulfonylamino)-4-hydroxyphenoxy]-3-[[(S)-1-hydroxy-3-(4-methoxyphenyl)-2-propyl]amino]-2-propanol

IR (KBr): 3380-3000, 1610, 1592, 1513, 1494, 1328, 1245, 1116 cm⁻¹

NMR (CD₃OD) δ: 2.60-3.10 (4H, m), 3.40-3.60 (3H, m), 3.73 (3H, s), 3.80-3.90 (2H, m), 3.95-4.10 (1H, m), 6.40-6.70 (2H, m), 6.80 (2H, d, J=8.4 Hz), 7.00 (1H, d, J=2.6 Hz), 7.10-7.20 (4H, m), 7.70-7.85 (2H, m)

MS (m/z): 521 (M+1)

(10) (S)-1-[3-(4-Chlorobenzenesulfonylamino)-4-hydroxyphenoxy]-3-[[(S)-1-hydroxy-3-(4-methoxyphenyl) -2-propyl]amino]-2-propanol

IR (KBr): 3380-3000, 1610, 1585, 1513, 1467, 1398, 1330, 1247, 1160, 1087, 1033 cm⁻¹

NMR (CD₃OD) δ: 2.50-3.00 (4H, m), 3.20-3.65 (3H, m), 3.72 (3H, s), 3.80 (2H, d, J=5.0 Hz), 4.00-4.10 (1H, m), 6.50-6.60 (2H, m), 6.80 (2H, d, J=8.5 Hz), 6.90 (1H, d, J=2.6 Hz), 7.10 (2H, d, J=8.5 Hz), 7.40 (2H, d, J=8.5 Hz), 7.70 (2H, d, J=8.5 Hz)

MS (m/z): 537 (M+1)

### EXAMPLE 23

The following compounds were obtained according to a similar manner to that of Example 2.

(1) (S)-1-[3-(4-Fluorobenzenesulfonylamino)-4-hydroxyphenoxy]-3-[[(S)-1-hydroxy-3-(4-hydroxyphenyl) -2-propyl]amino]-2-propanol

IR (KBr): 3380-3000, 1592, 1515, 1494, 1234, 1153, 1087, 1039 cm⁻¹

NMR (CD₃OD) δ: 2.50-3.00 (4H, m), 3.30-3.50 (3H, m), 3.60-3.70 (1H, m), 3.80 (2H, d, J=4.9 Hz), 3.90-4.00 (1H, m), 6.50-6.70 (4H, m), 6.90-7.20 (5H, m), 7.70-7.90 (2H, m)

MS (m/z): 507 (M+1)

(2) (S)-1-[3-(Benzenesulfonylamino)-4-hydroxyphenoxy]-3-[[(S)-1-hydroxy-3-(3-chloro-4-hydroxyphenyl)-2-propyl]amino]-2-propanol

IR (KBr): 3380-3000, 1612, 1589, 1513, 1326, 1494, 1234, 1155, 1089, 1049cm⁻¹

NMR (CD₃OD) δ: 2.70-3.10 (2H, m), 3.20-3.80 (5H, m), 3.90-4.00 (2H, m), 4.20-4.30 (1H, m), 6.50-6.60 (2H, m), 6.80-7.10 (3H, m), 7.28 (1H, d, J=1.7 Hz), 7.40-7.60 (3H, m), 7.70-7.80 (2H, m)

MS (m/z): 523 (M⁺)

(3) (S)-1-[3-(4-Fluorobenzenesulfonylamino)-4-hydroxyphenoxy]-3-[[(S)-1-hydroxy-3-(3-chloro-4-hydroxyphenyl)-2-propyl]amino]-2-propanol

IR (KBr): 3500-3000, 1592, 1513, 1455, 1328, 1155, 1089, 1052cm⁻¹

NMR (CD₃OD) δ: 2.80-3.05 (2H, m), 3.20-3.80 (5H, m), 3.90-4.00 (2H, m), 4.20-4.30 (1H, m), 6.60-6.70 (2H, m), 6.70-7.40 (7H, m), 7.70-7.90 (2H, m)

MS (m/z): 541 (M+1)

(4) (S)-1-[3-(4-Chlorobenzenesulfonylamino)-4-hydroxyphenoxy]-3-[[(S)-1-hydroxy-3-(3-chloro-4-hydroxyphenyl)-2-popyl]amino]-2-propanol

IR (KBr): 3500-3000, 1606, 1585, 1513, 1455, 1328, 1274, 1157, 1089, 1052 cm⁻¹

NMR (CD₃OD) δ: 3.20-3.30 (3H, m), 3.60-3.80 (4H, m), 3.90-4.00 (3H, m), 6.60-6.70 (3H, m), 6.90-7.00 (2H, m), 7.40-7.50 (3H, m), 7.70-7.80 (3H, m)

MS (m/z): 557 (M+1)

(5) N-[2-Hydroxy-5-[(2S)-2-hydroxy-3-[[ (1S)-2-hydroxy-1-(4-hydroxybenzyl)ethyl]amino]propoxy]phenyl]formamide

IR (KBr): 3300-3000, 1671, 1664, 1606, 1517, 1446, 1268, 1203, 1041 cm⁻¹

NMR (CD₃OD) δ: 2.70-3.00 (2H, m), 3.40-3.70 (2H, m), 3.80-4.00 (2H, m), 4.10-4.20 (1H, m), 6.60-6.90 (4H, m), 7.00-7.20 (2H, m), 7.70-7.80 (1H, m), 8.30 (1H, s)

MS (m/z): 377 (M+1)

### EXAMPLE 24

The following compound was obtained according to a similar manner to that of Example 19.

(R)-1-(4-Benzyloxy-3-nitrophenyl)-2-[N-benzyl-N-((S)-1-hydroxy-3-phenyl-2-propyl)amino]ethanol

MS (m/z): 513 (M+1)

### EXAMPLE 25

Under nitrogen, a solution of (S)-2-benzylamino-3-(4-hydroxyphenyl)-1-propanol (650 mg) and (R)-(4-benzyloxy-3-nitrophenyl)oxirane (686 mg) in ethanol (10 ml) was refluxed for 24 hours. The mixture was evaporated in vacuo. A mixture of the residue, potassium carbonate (420 mg), iodomethane (0.157 ml) and N,N-dimethylformamide (10 ml) was stirred at room temperature for 18 hours. The mixture was diluted with ethyl acetate, and insoluble materials were filtered off. The filtrate was evaporated in vacuo. The residue was dissolved in ethyl acetate, washed with aqueous saturated sodium bicarbonate solution and brine, dried over sodium sulfate, and evaporated in vacuo. The residue was purified by a column chromatography on silica gel (hexane:ethyl acetate=1:1) to give (2S)-2-[N-benzyl-N-[(2R)-2-[4-(benzyloxy)-3-nitrophenyl]-2-hydroxyethyl]amino]-3-(4-methoxyphenyl)-1-propanol (1.6 g) as a yellow foam.

MS (m/z): 543 (M+1)

### EXAMPLE 26

The following compound was obtained according to a similar manner to that of Example 25.

(2S)-2-[N-Benzyl-N-[(2S)-3-(4-benzyloxy-3-nitrophenoxy)-2-hydroxypropyl]amino]-3-(4-methoxyphenyl)-1-propanol

MS (m/z): 573 (M+1)

### EXAMPLE 27

The following compound was obtained according to a similar manner to that of Example 13.

(2S)-2-[N-Benzyl-N-[(2S)-3-(4-benzyloxy-3-nitrophenoxy)-2-hydroxypropyl]amino]-3-[3-chloro-4-(benzyloxy)phenyl]-1-propanol

MS (m/z): 683 (M+)

### EXAMPLE 28

The following compound was obtained according to a similar manner to that of Example 12.

(S)-3-[3-Amino-4-(benzyloxy)phenyl]-2-[N-benzyl-N-[(2R)-2-(3-chlorophenyl)-2-hydroxyethyl]amino]-1-propanol

MS (m/z): 517 (M+), 519 (M+2)

### EXAMPLE 29

A solution of N-[5-[(2S)-2-(benzylamino)-3-hydroxypropyl]-2-(benzyloxy)phenyl]-benzenesulfonamide (66 mg), 4-((2S)-oxiranylmethoxy)-1H-indole (24 mg) in ethanol (10 ml) was refluxed for 7 hours. The mixture was evaporated in vacuo. A mixture of the residue, 10% palladium on activated carbon (50% wet, 50 mg) and methanol (10.0 ml) was stirred at room temperature in the presence of hydrogen at an atmospheric pressure for 1 hour, and filtered. The filtrate was evaporated in vacuo. The residue was chromatographed (chloroform-methanol) over silica gel to afford N-[2-hydroxy-5-[(2S)-3-hydroxy-2-[[(2S)-2-hydroxy-3-(1H-indol-4-yloxy)propyl]amino]propyl]phenyl]-benzenesulfonamide (15 mg) as a colorless powder.

IR (KBr): 3480-3000, 1585, 1511, 1448, 1241, 1162, 1089 cm⁻¹

NMR (CD₃OD) δ: 2.50-3.20 (4H, m), 3.30-3.40 (1H, m), 3.50-3.70 (2H, m), 4.00-4.25 (3H, m), 6.40-6.90 (4H, m), 7.00-7.30 (4H, m), 7.40-7.60 (3H, m), 7.70-7.85 (2H, m)

MS (m/z): 512 (M+1)

### EXAMPLE 30

The following compounds were obtained according to a similar manner to that of Example 29.

(1) N-[5-[(2S)-2-[[(2S)-3-(9H-Carbazol-4-yloxy)-2-hydroxypropyl]amino]-3-hydroxypropyl]-2-hydroxyphenyl]-benzenesulfonamide

IR (KBr): 3480-3000, 1606, 1585, 1511, 1471, 1450, 1330, 1162, 1093 cm⁻¹

NMR (CD₃OD) δ: 2.50-3.10 (4H, m), 3.30-3.60 (3H, m), 4.20-4.40 (3H, m), 6.60-6.90 (3H, m), 7.10-7.50 (9H, m), 7.70-7.80 (2H, m), 8.30 (1H, d, J=7.8 Hz)

MS (m/z): 561 (M+1)

(2) N-[5-[(2S)-2-[[(2S)-3-(4-Fluorophenoxy)-2-hydroxypropyl]amino]-3-hydroxypropyl]-2-hydroxyphenyl]-benzenesulfonamide

IR (KBr): 3480-3000, 1602, 1452, 1400, 1326, 1288, 1207, 1162, 1091 cm⁻¹

NMR (CD₃OD) δ: 2.40-3.00 (5H, m), 3.30-3.60 (2H, m), 3.90-4.10 (3H, m), 6.60 (1H, d, J=8.2 Hz), 6.70-7.20 (6H, m), 7.40-7.60 (3H, m), 7.70-7.90 (2H, m)

MS (m/z): 491 (M+1)

### EXAMPLE 31

The following compounds were obtained according to a similar manner to that of Example 5.

(1) 4-[(2S)-3-Hydroxy-2-[((2S)-2-hydroxy-3-phenoxypropyl)amino]phenyl trifluoromethanesulfonate

NMR (CD₃OD) δ: 2.60-3.00 (5H, m), 3.30-3.60 (2H, m), 3.90-4.10 (3H, m), 6.80-7.50 (9H, m)

MS (m/z): 450 (M+1)

(2) (2S)-2-[((2S)-2-Hydroxy-3-phenoxypropyl)amino]-3-[4-(trifluoromethyl)phenyl]-1-propanol

NMR (CD₃OD) δ: 2.60-3.00 (5H, m), 3.40-3.60 (2H, m), 3.85-4.05 (3H, m), 6.80-7.00 (3H, m), 7.20-7.30 (2H, m), 7.40-7.70 (4H, m)

MS (m/z): 370 (M+1)

(3) 2-Chloro-4-[(2S)-3-hydroxy-2-[((2S)-2-hydroxy-3-phenoxypropyl)amino]propyl]phenol hydrochloride

IR (KBr): 3500-3200, 1752, 1594, 1504, 1290, 1240 cm⁻¹

NMR (D₂O) δ: 2.95-3.10 (2H, m), 3.30-3.40 (2H, m), 3.60-3.90 (3H, m), 4.10 (2H, d, J=4.2 Hz), 4.30-4.40 (1H, m), 6.90-7.20 (5H, m), 7.30-7.50 (3H, m)

MS (m/z): 352 (M+1)

(4) 4-[(2R)-3-Hydroxy-2-[((2S)-2-hydroxy-3-phenoxypropyl)amino]propyl]phenol hydrochloride

IR (KBr): 3500-3200, 1594, 1513, 1455, 1240 cm⁻¹

NMR (D₃O) δ: 2.95-3.50 (4H, m), 3.70-4.00 (3H, m), 4.10-4.20 (2H, m), 4.20-4.30 (1H, m), 6.90-7.50 (9H, m)

MS (m/z): 318 (M+1)

(5) 2-Fluoro-4-[(2S)-3-hydroxy-2-[((2S)-2-hydroxy-3-phenoxypropyl)amino]propyl]phenol hydrochloride

IR (KBr): 3500-3000, 1596, 1536, 1455, 1294, 1241, 1116 cm⁻¹

NMR (CD₃OD) δ: 2.90-3.10 (2H, m), 3.30-3.40 (2H, m), 3.60-3.90 (3H, m), 4.10-4.20 (2H, m), 4.30-4.40 (1H, m), 6.90-7.20 (6H, m), 7.50-7.60 (2H, m)

MS (m/z): 336 (M+1)

### EXAMPLE 32

The following compounds were obtained according to a similar manner to that of Example 2.

(1) (S)-1-Phenoxy-3-[[(S)-1-hydroxy-3-[3-(methanesulfonylamino)-4-hydroxyphenyl]-2-propyl]amino]-2-propanol

IR (KBr): 3500-3000, 1650, 1558, 1521, 1513, 1455, 1392, 1315, 1147, 1039 cm⁻¹

NMR (CD₃OD) δ: 2.60-2.90 (8H, m), 3.30-3.70 (2H, m), 3.90-4.10 (3H, m), 6.80-7.00 (5H, m), 7.20-7.30 (2H, m)

MS (m/z): 411 (M+1)

(2) 4-Chloro-N-[2-hydroxy-5-[(2S)-3-hydroxy-2-[((2S)-2-hydroxy-3-phenoxypropyl)amino]propyl]phenyl]-benzenesulfonamide

NMR (CD₃OD) δ: 2.80-3.80 (7H, m), 4.00-4.15 (2H, m), 4.20-4.30 (1H, m), 6.60-6.70 (1H, m), 6.90-7.10 (4H, m), 7.30-7.35 (2H, m), 7.40 (2H, J=8.6 Hz), 7.70 (2H, d, J=8.6 Hz)

MS (m/z): 507 (M+), 509 (M+2)

(3) Methyl 2-hydroxy-5-[(2S)-3-hydroxy-2-[((2S)-2-hydroxy-3-phenoxypropyl)amino]propyl]phenylcarbamate

IR (KBr): 3380-3000, 1737, 1606, 1544, 1446, 1247, 1218, 1062, 1029 cm⁻¹

NMR (CD₃OD) δ: 2.60-3.10 (5H, m), 3.40-3.55 (2H, m), 3.73 (3H), 3.90 (2H, d, J=4.8 Hz), 4.00-4.10 (1H, m), 6.75-6.80 (2H, m), 6.90-7.00 (3H, m), 7.20-7.30 (2H, m), 7.60-7.70 (1H, m)

MS (m/z): 391 (M+1)

(4) N-[5-[(2S)-2-[[(2R)-2-(3-Chlorophenyl)-2-hydroxyethyl]amino]-3-hydroxypropyl]-2-hydroxyphenyl]-benzenesulfonamide

IR (KBr): 3380-3000, 1592, 1446, 1407, 1328, 1164, 1089 cm⁻¹

NMR (CD₃OD) δ: 2.70-3.20 (7H, m), 3.30-3.50 (4H, m), 3.60-3.80 (2H, m), 6.60-6.70 (1H, m), 6.80-6.90 (1H, m), 7.20-7.60 (8H, m), 7.70-7.80 (2H, m)

MS (m/z): 477 (M+1)

(5) 4-Fluoro-N-[2-hydroxy-5-[(2S)-3-hydroxy-2-[((2S)-2-hydroxy-phenoxypropyl)amino]propyl]phenyl]-benzenesulfonamide

IR (KBr): 3480-3000, 1594, 1513, 1494, 1402, 1240, 1330, 1153, 1170 cm⁻¹

NMR (CD₃OD) δ: 2.40-3.00 (5H, m), 3.30-3.70 (2H, m), 3.90-4.10 (3H, m), 6.60-6.70 (1H, m), 6.80-7.00 (4H, m), 7.10-7.40 (5H, m), 7.70-7.90 (2H, m)

MS (m/z): 491 (M+1)

### EXAMPLE 33

The following compounds were obtained according to a similar manner to that of Example 1.

(1) Methyl 4-[(2S)-3-hydroxy-2-[((2S)-2-hydroxy-3-phenoxypropyl)amino]propyl]benzoate

IR (KBr): 3480-3000, 1724, 1604, 1594, 1496, 1278, 1245, 1108, 1043 cm⁻¹

NMR (CD₃OD) δ: 2.90-3.10 (5H, m), 3.40-3.60 (2H, m), 3.80-4.10 (6H, m), 6.80-7.40 (8H, m), 7.90-8.00 (2H, m)

MS (m/z): 360 (M+1)

(2) 1-[4-[(2S)-3-Hydroxy-2-[((2S)-2-hydroxy-3-phenoxypropyl)amino]propyl]phenyl]ethanone

IR (KBr): 3480-3000, 1687, 1602, 1446, 1355, 1303, 1245, 1174, 1039 cm⁻¹

NMR (CD₃OD) δ: 2.56 (3H, s), 2.60-3.00 (5H, m), 3.40-3.60 (2H, m), 3.90-4.10 (3H, m), 6.90-7.00 (2H, m), 7.20-7.50 (6H, m), 7.90-8.00 (1H, m)

MS (m/z): 344 (M+1)

### EXAMPLE 34

4-Chlorobenzenesulfonyl chloride (87 mg) was added to a solution of (2S)-3-[3-amino-4-(benzyloxy)phenyl]-2-[N-benzyl-N-((2S)-2-hydroxy-3-phenoxypropyl)amino]-1-propanol (230 mg) and pyridine (0.5 ml) in dichloromethane (5 ml) at room temperature for 18 hours. To this one was added aqueous saturated solution of sodium bicarbonate (5.0 ml). The mixture was stirred at the same temperature for 1 hour, and which was dissolved in ethyl acetate, washed with aqueous saturated sodium bicarbonate solution and brine, dried over sodium sulfate, and evaporated in vacuo to give N-[2-(benzyloxy)-5-[(2S)-3-hydroxy-2-[N-((2S)-2-hydroxy-3-phenoxypropyl)-N-benzylamino]propyl]phenyl]-benzenesulfonamide (210 mg) as a yellow foam.

MS (m/z): 653 (M+1)

### EXAMPLE 35

Iodomethane (50 mg) was added to a solution of N-[2-(benzyloxy)-5-[(2S)-3-hydroxy-2-[N-benzyl-N-((2S)-2-hydroxy-3-phenoxypropyl)amino]propyl]phenyl]-benzenesulfonamide (210 mg) and pyridine (0.1 ml) in dichloromethane (5 ml) at room temperature for 18 hours. To this one was added aqueous saturated solution of sodium bicarbonate (5.0 ml). The mixture was stirred at the same temperature for 1 hour, and which was dissolved in ethyl acetate, washed with aqueous saturated sodium bicarbonate solution and brine, dried over sodium sulfate, and evaporated in vacuo to give N-[2-hydroxy-5-[(2S)-3-hydroxy-2-[((2S)-2-hydroxy-3-phenoxypropyl)amino]propyl]phenyl]-N-methyl-benzenesulfonamide (50 mg) as a yellow foam.

IR (KBr): 3480-3000, 1596, 1511, 1454, 1338, 1241, 1039 cm⁻¹

NMR (CD₃OD) δ: 2.40-3.00 (5H, m), 3.17 (3H, s), 3.40-3.70 (2H, m), 3.90-4.10 (3H, m), 6.70-6.80 (2H, m), 6.90-7.10 (4H, m), 7.30-7.40 (2H, m), 7.50-7.80 (5H, m)

MS (m/z): 487 (M+1)

### EXAMPLE 36

A mixture of 4-[(2S)-2-[N-benzyl-N-((2S)-2-hydroxy-3-phenoxypropyl)amino]-3-hydroxypropyl]phenyl trifluoro-methanesulfonate (7.0 g) and tetrakis(triphenylphosphine)-palladium (76 mg), 1,3-bis(diphenylphosphino)propane (150 mg), triethylamine (3.62 ml), N,N-dimethylformamide (35 ml) and methanol (5.0 ml) was stirred at 70° C. in the presence of carbon monooxide at an atmospheric pressure for 4 hours. The mixture was dissolved in ethyl acetate, washed with aqueous saturated sodium bicarbonate solution and brine, dried over sodium sulfate, and evaporated in vacuo to give methyl 4-[(2S)-2-(N-benzyl-N-((2S)-2-hydroxy-3-phenoxypropyl)amino]-3-hydroxypropyl]benzoate (5.13 g) as a colorless foam.

NMR (CD₃Cl) δ: 2.60-3.10 (6H, m), 3.20-3.30 (1H, m), 3.50-4.10 (9H, m), 6.90-7.05 (3H, m), 7.20-7.40 (9H, m), 7.80-8.00 (2H, m).

MS (m/z): 450 (M+1)

### EXAMPLE 37

To a solution of methyl 4-[(2S)-2-[N-benzyl-N-((2S)-2-hydroxy-3-phenoxypropyl)amino]-3-hydroxypropyl]benzoate (100 mg) in 1N hydrogen chloride in tetrahydrofuran (5 ml) was added lithium aluminum hydride (25 mg) on ice-cooling and stirred at room temperature for 1 hour. To the mixture was added water (1 ml) on ice-cooling and dissolved in ethyl acetate, washed with aqueous saturated sodium bicarbonate solution and brine, dried over sodium sulfate and evaporated in vacuo. A mixture of the residue, 10% palladium on activated carbon (50% wet, 10 mg) and methanol (5.0 ml) was stirred at room temperature in the presence of hydrogen at an atmospheric pressure for 1 hour, and filtered. The filtrate was evaporated in vacuo to give (2S)-3-[4-(hydroxymethyl)phenyl]-2-[((2S)-2-hydroxy-3-phenoxypropyl)amino]-1-propanol (30 mg) as a colorless foam.

IR (KBr): 3480-3000, 1594, 1496, 1446, 1245, 1139, 1060, 1039 cm⁻¹

NMR (CD₃OD) δ: 2.60-3.10 (5H, m), 3.40-3.60 (2H, m), 3.90-4.10 (3H, m), 4.55 (2H, s), 6.90-7.00 (3H, m), 7.20-7.40 (6H, m)

MS (m/z): 332 (M+1)

### EXAMPLE 38

A solution of methyl 4-[(2S)-2-[N-benzyl-N-((2S)-2-hydroxy-3-phenoxypropyl)amino]-3-hydroxypropyl]benzoate (400 mg) in 1N sodium hydroxide (4 ml), tetrahydrofuran (3 ml) was refluxed for 7 hours. The mixture was dissolved in ethanol acetate, washed with aqueous saturated sodium bicarbonate solution and brine, dried over sodium sulfate and evaporated in vacuo. A mixture of the residue, 10% palladium on activated carbon (50% wet, 10 mg) and methanol (5.0 ml) was stirred at room temperature in the presence of hydrogen at an atmospheric pressure for 1 hour, and filtered. The filtrate was evaporated in vacuo to give 4-[(2S)-3-hydroxy-2-[((2S)-2-hydroxy-3-phenoxypropyl)amino]propyl]-benzoic acid (90 mg) as a colorless powder.

IR (KBr): 3480-3000, 1594, 1548, 1457, 1384, 1241, 1174, 1043 cm⁻¹

NMR (CD₃OD) δ: 2.90-3.80 (5H, m), 4.00-4.10 (2H, m), 4.20-4.30 (1H, m), 6.90-7.05 (3H, m), 7.20-7.40 (4H, m), 7.80-8.00 (2H, m)

MS (m/z): 346 (M+1)

### EXAMPLE 39

A solution of methyl 4-[(2S)-2-[N-benzyl-N-((2S)-2-hydroxy-3-phenoxypropyl)amino]-3-hydroxypropyl]benzoate (400 mg) in 1N sodium hydroxide (4 ml), tetrahydrofuran (3 ml) was refluxed for 7 hours. The mixture was dissolved in ethyl acetate, washed with aqueous saturated sodium bicarbonate solution and brine, dried over sodium sulfate and evaporated in vacuo. A solution of the residue, dimethylamine hydrochloride (50 mg), 1-[3-(dimethylamino)propyl]-3-ethylcarbodiimide (90 mg) and 1-hydroxybenzotriazole hydrate (50 mg) in dichloromethane (5 ml) was stirred for 3 hours. The mixture was dissolved in ethyl acetate, washed with aqueous saturated sodium bicarbonate solution and brine, dried over sodium sulfate and evaporated in vacuo. A mixture of the residue, 10% palladium on activated carbon (50% wet, 10 mg) and methanol (5.0 ml) was stirred at room temperature in the presence of hydrogen at an atmospheric pressure for 1 hour, and filtered. The filtrate was evaporated in vacuo to give 4-[(2S)-3-hydroxy-2-[((2S)-2-hydroxy-3-phenoxypropyl)amino]propyl]-N,N-dimethylbenzamide (30 mg) as a colorless foam.

IR (KBr): 3500-3200, 1720, 1604, 1496, 1446, 1407, 1243 cm⁻¹

NMR (CD₃OD) δ: 2.70-3.10 (8H, m), 3.30-3.70 (2H, m), 3.90-4.10 (3H, m), 6.80-7.00 (3H, m), 7.20-7.40 (6H, m)

MS (m/z): 373 (M+1)

### EXAMPLE 40

A mixture of 4-[(2S)-2-[N-benzyl-N-((2S)-2-hydroxy-3-phenoxypropyl)amino]-3-hydroxypropyl]phenyl trifluoromethanesulfonate (5.0 g) and tetrakis(triphenylphosphine)palladium (52 mg), 1,3-bis(diphenylphosphino)propane (107 mg), triethylamine (2.6 ml), N,N-dimethylformamide (50 ml) and butyl vinyl ether (5.9 ml) was stirred at 80° C. for 1 hour. To the mixture was added 1N hydrogen chloride (25 ml) under ice-cooling and stirred for 0.5 hour at the same temperature. The mixture was dissolved in ethyl acetate, washed with aqueous saturated sodium bicarbonate solution and brine, dried over sodium sulfate, and evaporated in vacuo to give 1-[4-[(2S)-2-[N-benzyl-N-((2S)-2-hydroxy-3-phenoxypropyl)amino]-3-hydroxypropyl]phenyl]ethanone (2.71 g) as a colorless foam.

NMR (CD₃Cl) δ: 2.58 (3H, s), 2.60-3.20 (6H, m), 3.50-4.10 (7H, m), 6.90-7.05 (3H, m), 7.20-7.40 (9H, m), 7.80-8.00 (2H, m)

MS (m/z): 434 (M+1)

### EXAMPLE 41

A solution of 1-[4-[(2S)-2-[N-benzyl-N-((2S)-2-hydroxy-3-phenoxypropyl) amino]-3-hydroxypropyl]phenyl]ethanone (200 mg), thallium nitrate trihydrate (246 mg), perchloric acid (0.55 ml), dioxane (1 ml) and methanol (3 ml) was stirred at room temperature for 18 hours. The mixture was dissolved in ethyl acetate, washed with aqueous saturated sodium bicarbonate solution and brine, dried over sodium sulfate and evaporated in vacuo. A mixture of the residue, 10% palladium on activated carbon (50% wet, 10 mg) and methanol (5.0 ml) was stirred at room temperature in the presence of hydrogen at an atmospheric pressure for 1 hour, and filtered. The filtrate was evaporated in vacuo to give [4-[(2S)-3-hydroxy-2-[((2S)-2-hydroxy-3-phenoxypropyl) amino]propyl]-phenyl]acetic acid (50 mg) as a colorless foam.

IR (KBr): 3480-3000, 1589, 1446, 1241 cm⁻¹

NMR (CD₃OD) δ: 2.80-3.10 (2H, m), 3.30-3.60 (6H, m), 3.70-3.80 (1H, m), 3.90-4.10 (2H, m), 4.20-4.30 (1H, m), 6.80-7.00 (3H, m), 7.10-7.40 (6H, m)

MS (m/z): 360 (M+1)

### EXAMPLE 42

The following compound was obtained according to a similar manner to that of Example 29.

4-[(2S)-2-[[(2S)-3-(9H-Carbazol-4-yloxy)-2-hydroxypropyl]amino]-3-hydroxypropyl]phenol

IR (KBr): 3380-3000, 1592, 1496, 1407, 1328, 1153, 1039 cm⁻¹

NMR (CD₃OD) δ: 2.80-3.80 (7H, m), 4.00-4.15 (2H, m), 4.20-4.30 (1H, m), 6.60-6.70 (1H, m), 6.90-7.10 (4H, m), 7.30-7.35 (2H, m), 7.40 (2H, J=8.6 Hz), 7.70 (2H, d, J=8.6 Hz)

MS (m/z): 507 (M+), 509 (M+2)

1. A compound of the formula [I]: 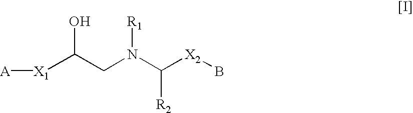
wherein
X₁ is bond or —O(CH₂)ₘ— (in which m is an integral number of 1, 2 or 3);
X₂ is bond, —(CH₂)ₙ— or —CH₂O— (in which n is an integral number of 1, 2 or 3);
R₁ is hydrogen or an amino protective group;
R₂ is hydroxy(lower)alkyl or (lower)alkoxy(lower)alkyl;
A is phenyl, pyridyl, indolyl or carbazolyl, each of which may be substituted with one or two substituent(s) selected from the group consisting of halogen, hydroxy, nitro, lower alkoxy, phenyl(lower)alkoxy, optionally substituted lower alkyl and optionally substituted amino; and
B is phenyl or pyridyl, each of which may be substituted with one or two substituent(s) selected from the group consisting of halogen, hydroxy, nitro, lower alkanoyl carboxy, (halo(lower)alkyl)sulfonyloxy, optionally substituted amino, optionally substituted lower alkyl, optionally substituted ureido, optionally substituted carbamoyl, (lower)alkoxycarbonyl and lower alkoxy optionally substituted with lower alkoxy, carboxy or phenyl;
(i) provided that when X₁ is —O(CH₂)ₘ— (in which m is an integral number of 1);
X₂ is —(CH₂)ₙ— (in which n is an integral number of 1);
R₁ is hydrogen;
R₂ is hydroxymethyl; and
A is phenyl or pyridyl, each of which may be substituted with one or two substituent(s) selected from the group consisting of halogen, lower alkoxy and lower alkyl; or indolyl,
then B is not phenyl which may be substituted with one or two substituent(s) selected from the group consisting of halogen and lower alkoxy optionally substituted with lower alkoxy or carboxy;
(ii) provided that when X₁ is bond;
X₂ is —(CH₂)ₙ— (in which n is an integral number of 1);
R₁ is hydrogen;
R₂ is hydroxymethyl; and
A is phenyl or pyridyl, each of which may be substituted with one or two substituent(s) selected from the group consisting of halogen and lower alkyl,
then B is not phenyl which may be substituted with one or two substituent(s) selected from the group consisting of halogen, hydroxy, carboxy, nitro and lower alkoxy optionally substituted with lower alkoxy or carboxy;
or a salt thereof.

2. A compound of claim 1,
wherein
X₁ is bond or —O(CH₂)ₘ— (in which m is an integral number of 1);
X₂ is —(CH₂)ₙ— (in which n is an integral number of 1 or 2);
R₁ is hydrogen; and
R₂ is hydroxy(lower)alkyl.

3. A compound of claim 2,
wherein
A is phenyl, pyridyl, indolyl or carbazolyl, each of which may be substituted with one or two substituent(s) selected from the group consisting of halogen, hydroxy, nitro, lower alkoxy, phenyl(lower)alkoxy, lower alkyl, hydroxy(lower)alkyl, (lower)alkoxycarbonyl(lower)alkyl, amino, phenylsulfonylamino optionally substituted with halogen, (lower)alkylsulfonylamino and (lower)alkanoylamino; and
B is phenyl or pyridyl, each of which may be substituted with one or two substituent(s) selected from the group consisting of halogen; hydroxy; nitro; lower alkanoyl; carboxy; (trifluoromethyl)sulfonyloxy; lower alkyl; halo(lower)alkyl; hydroxy(lower)alkyl; carboxy(lower)alkyl; (lower)alkoxycarbonyl(lower)alkyl; amino; (lower)alkoxycarbonylamino; (lower)alkylsulfonylamino; phenylsulfonylamino optionally substituted with halogen; N-(lower alkyl)-N-(phenylsulfonyl)amino optionally substituted with halogen; (lower)alkylsulfonylureido; (lower)alkoxycarbonyl; carbamoyl which may be substituted with one or two substituent(s) selected from the group consisting of lower alkyl, lower alkoxy, (lower)alkylsulfonyl, phenyl and phenylsulfonyl; and lower alkoxy optionally substituted with lower alkoxy, carboxy or phenyl.

4. A compound of claim 3,
wherein
A is phenyl, pyridyl or carbazolyl, each of which may be substituted with one or two substituent(s) selected from the group consisting of hydroxy, hydroxy(lower)alkyl, nitro, amino, phenylsulfonylamino optionally substituted with halogen, (lower)alkylsulfonylamino and formylamino; and
B is phenyl or pyridyl, each of which may be substituted with one or two substituent(s) selected from the group consisting of halogen, hydroxy, lower alkanoyl, carboxy, (trifluoromethyl)sulfonyloxy, hydroxy(lower)alkyl, carboxy(lower)alkyl, (lower)alkoxycarbonyl(lower)alkyl, amino, (lower)alkoxycarbonylamino, (lower)alkylsulfonylamino, phenylsulfonylamino optionally substituted with halogen, N-(lower alkyl)-N-(phenylsulfonyl)amino, di(lower alkyl)carbamoyl and lower alkoxy optionally substituted with lower alkoxy or phenyl.

5. A compound of claim 4,
wherein
X₁ is —OCH₂—;
X₂ is —CH₂—;
A is phenyl which may be substituted with one or two substituent(s) selected from the group consisting of hydroxy, hydroxy(lower)alkyl, nitro, amino, phenylsulfonylamino optionally substituted with halogen, (lower)alkylsulfonylamino and formylamino; and
B is phenyl which may be substituted with one or two substituent(s) selected from the group consisting of halogen, hydroxy, lower alkanoyl, carboxy, (trifluoromethyl)sulfonyloxy, hydroxy(lower)alkyl, carboxy(lower)alkyl, (lower)alkoxycarbonyl(lower)alkyl, amino, (lower)alkoxycarbonylamino, (lower)alkylsulfonylamino, phenylsulfonylamino optionally substituted with halogen, N-(lower alkyl)-N-(phenylsulfonyl)amino, di(lower alkyl)carbamoyl and lower alkoxy optionally substituted with lower alkoxy or phenyl.

6. A compound of claim 5,
wherein
R₂ is hydroxymethyl;
A is phenyl substituted with hydroxy and phenylsulfonylamino optionally substituted with halogen; and
B is phenyl substituted with hydroxy or lower alkoxy.

7. A compound of claim 4,
wherein
X₁ is bond;
R₂ is hydroxymethyl;
A is phenyl which may have one or two substituent(s) selected from the group consisting of hydroxy, hydroxy(lower)alkylf amino, (lower)alkylsulfonylamino, phenylsulfonylamino which may be substituted with halogen and formylamino; and
B is phenyl which may have one or two substituent(s) selected from the group consisting of hydroxy, halogen and lower alkoxy.

8. A process for preparing a compound of the formula [I]: 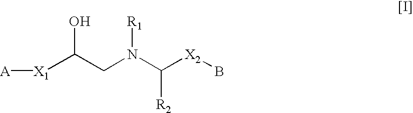
wherein
X₁, X₂, R₁, R₂, A and B are each as defined in claim 1,
which comprises,
(i) reacting a compound of the following formula [II]: 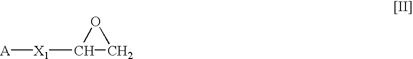
wherein
A and X₁ are each as defined above, with a compound of the following formula [III]: 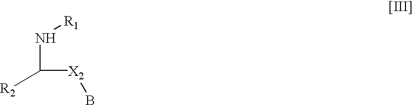
wherein
R₁, R₂, X₂ and B are each as defined in claim 1,
or a salt thereof to give a compound of the following formula [I]: 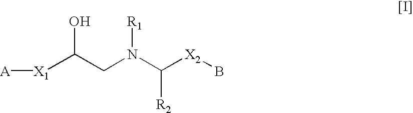
wherein
A, B, R₁, R₂, X₁ and X₂ are each as defined above,
or a salt thereof, or
(ii) subjecting a compound of the following formula [Ia]: 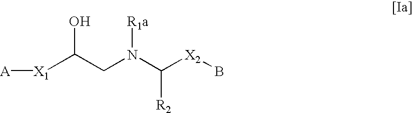
wherein
A, B, R₂, X₁ and X₂ are each as defined above, and
R₁a is an amino protective group,
or a salt thereof, to deprotection of the amino protective group, to give a compound of the following formula [Ib]: 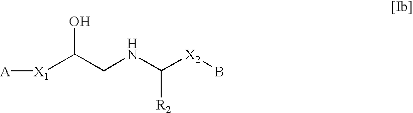
wherein
A, B, R₂, X₁ and X₂ are each as defined above,
or a salt thereof,
(iii) subjecting a compound of the following formula [Ic]: 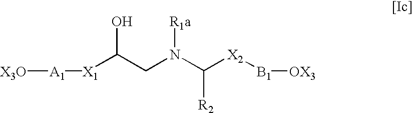
wherein
R₁a, R₂, X₁ and X₂ are each as defined above,
A₁ is phenyl, pyridyl or indolyl,
B₁ is phenyl or pyridyl, and
X₃ is a hydroxy protective group,
or a salt thereof, to deprotection of the hydroxy protective group and the amino protective group, to give a compound of the following formula [Id]: 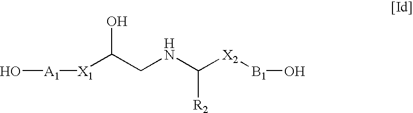
wherein
R₂ X₁, X₂, A₁ and B₁ are each as defined above,
or a salt thereof,
(iv) subjecting a compound of the following formula [Ie]: 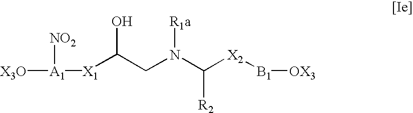
wherein
R₁a, R₂, X₁, X₂, X₃, A₁ and B₁ are each as defined above,
or a salt thereof, to reduction reaction to give a compound of the following formula [If]: 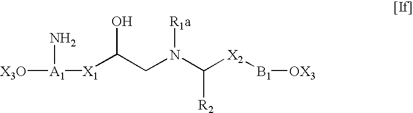
wherein
R₁a, R₂, X₁, X₂, X₃, A₁ and B₁ are each as defined above,
or a salt thereof, and then reacting the compound thus obtained with a compound of the following formula [Ig]:
RO₂SX₄ [Ig]
wherein
R is lower alkyl or phenyl optionally substituted with halogen, and
X₄ is halogen,
to give a compound of the following formula [Ih]: 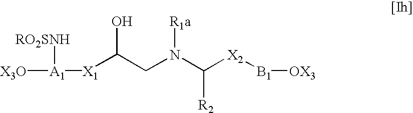
wherein
R₁a, R₂, X₁, X₂, X₃, R, A₁ and B₁ are each as defined above,
or a salt thereof.

9. A pharmaceutical composition which comprises, as an active ingredient, a compound of claim 1 or a pharmaceutically acceptable salt thereof in admixture with pharmaceutically acceptable carriers or excipients.

10. Use of a compound of claim 1 or a pharmaceutically acceptable salt for the manufacture of a medicament.

11. A compound of claim 1 or pharmaceutically acceptable salt thereof for use as a medicament.

12. A compound of claim 1 or pharmaceutically acceptable salt thereof for use as selective β₃ adrenergic receptor agonist.

13. A method for the prophylactic and/or the therapeutic treatment of pollakiuria, urinary incontinence, a wasting condition, or emaciation which comprises administering a compound of claim 1 or a pharmaceutically acceptable salt thereof to a human being or an animal.

14. A prophylactic or therapeutic agent for pollakiuria or urinary incontinence, which comprises (S)-4-[2-hydroxy-3-[[2-[4-(5-carbamoyl-2-pyridyloxy)phenyl]-1,1-dimethylethyl]amino]propoxy]carbazole or hydrochloride salt thereof.

15. A method for the prophylactic and/or therapeutic treatment of pollakiuria or urinary incontinence, which comprises administering (S)-4-[2-hydroxy-3-[[2-[4-(5-carbamoyl-2-pyridyloxy)phenyl]-1,1-dimethylethyl]-amino]propoxy]carbazole or hydrochloride salt thereof.

16. Use of (S)-4-[2-hydroxy-3-[[2-[4-(5-carbamoyl-2-pyridyloxy)phenyl]-1,1-dimethylethyl]amino]propoxy]-carbazole or hydrochloride salt thereof for manufacturing a medicament for the prophylactic and/or therapeutic treatment of pollakiuria or urinary incontinence.
